(19) United States
(12) Patent Application Publication
(10) Pub. No.: US 2014/0180307 A1
(43) Pub. Date: Jun. 26, 2014

(54) ELECTROSTIMULATION IN TREATING CEREBROVASCULAR CONDITIONS

(71) Applicants: Alon Shalev, Ra'anana (IL); Ronnie Levy, Kochav-Yair (IL); Yiftach Beinart, Hod Hasharon (IL)

(72) Inventors: Alon Shalev, Ra'anana (IL); Ronnie Levy, Kochav-Yair (IL); Yiftach Beinart, Hod Hasharon (IL)

(21) Appl. No.: 14/235,470

(22) PCT Filed: Aug. 2, 2012
(86) PCT No.: PCT/IL2012/000290

§ 371 Date: Jan. 28, 2014

Related U.S. Application Data
(60) Provisional application No. 61/514,066, filed on Aug. 2, 2011.
(60) Provisional application No. 61/514,067, filed on Aug. 2, 2011.
(60) Provisional application No. 61/514,082, filed on Aug. 2, 2011.
(60) Provisional application No. 61/514,523, filed on Aug. 3, 2011.
(60) Provisional application No. 61/514,520, filed on Aug. 3, 2011.

(51) Int. Cl. A61N 1/36; A61N 1/378

(52) U.S. Cl. CPC A61N 1/36103; A61N 1/3787
USPC 606/129; 607/116; 607/60

(57) ABSTRACT

A system for treating a medical condition in a living body, comprising two subsystems, an implant subsystem and an electrical stimulation unit subsystem. The implant subsystem comprises at least one electrostimulation module, contains at least one electrically conductive electrode and, preferably, an anchoring member. The electrical stimulation unit, adapted for producing and controlling electrical waveforms, is connected to the electrodes. The implant subsystem is implanted adjacent to at least one of the following structures: the carotid sinus nerve, aortic nerve, common carotid artery, external carotid artery, internal carotid artery, carotid artery bifurcation, carotid body, aortic body or aortic arch receptors. The electrical stimulation unit is maintained outside the patient's body and is adapted to program, generate, control and deliver the electrical waveform via a wired or a wireless connection to the implant subsystem, thereby stimulating the structure it is adjacent to and treating the medical condition.

### FIELD OF THE INVENTION

This invention relates to a medical apparatus and a method for the treatment of physiological disorders such as cerebral brain vasospasm, ischemia and brain injury. More particularly this invention relates to the stimulation of at least one selected from a group consisting of: chemoreceptors, baroreceptors and aortic arch receptors in order induce vasodilatation in blood vessels of the brain.

### BACKGROUND OF THE INVENTION

### Cardiovascular Regulation of Blood Pressure

In human physiology, several negative feedback systems control blood pressure by adjusting heart rate, stroke volume, systemic vascular resistance and blood volume. Some allow rapid adjustment of blood pressure to cope with sudden changes such as the drop in cerebral blood pressure when rising up. Others act more slowly to provide long-term regulation of blood pressure. Even if blood pressure is steady, there may be a need to change the distribution of blood flow, which is accomplished mainly by altering the diameter of arterioles. Groups of neurons scattered within the medulla of the brain stem regulate heart rate, contractility of the ventricles, and blood vessel diameter. As a whole, this region is known as the cardiovascular center, which contains both a cardiostimulatory center and a cardioinhibitory center. The cardiovascular center includes a vasomotor center, which includes vasoconstriction and vasodilatation centers that influence blood vessel diameter. Since these clusters of neurons communicate with one another, function together, and are not clearly separated anatomically, they are usually taken as a group. The cardiovascular center receives input both from higher brain regions and from sensory receptors. Nerve impulses descend from higher brain regions including the cerebral cortex, limbic system and hypothalamus to affect the cardiovascular center. The two main types of sensory receptors that provide input to the cardiovascular center are baroreceptors and chemoreceptors. Baroreceptors are important pressure-sensitive sensory neurons that monitor stretching of the walls of blood vessels and the atria. Chemoreceptors monitor blood acidity, carbon dioxide level and oxygen level.

Output from the cardiovascular center flows along sympathetic and parasympathetic fibers of the autonomic nervous system. Sympathetic stimulation of the heart increases heart rate and contractility. Sympathetic impulses reach the heart via the cardiac accelerator nerves. Parasympathetic stimulation, conveyed along the vagus nerves, decreases heart rate. The cardiovascular center also continually sends impulses to smooth muscle in blood vessel walls via sympathetic fibers called vasomotor nerves. Thus autonomic control of the heart is the result of opposing sympathetic (stimulatory) and parasympathetic (inhibitory) influences. Autonomic control of blood vessels, on the other hand, is mediated exclusively by the sympathetic division of the autonomic nervous system.

In the smooth muscle of most small arteries and arterioles, sympathetic stimulation causes vasoconstriction and thus raises blood pressure. This is due to activation of alpha-adrenergic receptors for norepinephrine and epinephrine in the vascular smooth muscle. In skeletal muscle and the heart, the smooth muscle of blood vessels displays beta-adrenergic receptors instead, and sympathetic stimulation causes vasodilatation rather than vasoconstriction. In addition, some of the sympathetic fibers to blood vessels in skeletal muscle are cholinergic; they release acetylcholine, which causes vasodilatation.

### Neural Regulation of Blood Pressure

Nerve cells capable of responding to changes in pressure or stretch are called baroreceptors. Baroreceptors in the walls of the arteries, veins, and right atrium monitor blood pressure and participate in several negative feedback systems that contribute to blood pressure control. The three most important baroreceptor negative feedback systems are the aortic reflex, carotid sinus reflex and right heart reflex.

A carotid sinus reflex is concerned with maintaining normal blood pressure in the brain and is initiated by baroreceptors in the wall of a carotid sinus. A carotid sinus is a small widening of the internal carotid artery just above the bifurcation of the common carotid artery. Any increase in blood pressure stretches the wall of the aorta and a carotid sinus, and the stretching stimulates the baroreceptors. A carotid sinus nerve, which is an afferent nerve tract that originates in carotid sinus baroreceptors, converges with the glossopharyngeal nerve, passes through the jugular foramen, reaches the rostral end of the medulla, and continues to the cardiovascular center. When an increase in aortic or carotid artery pressures is detected in this manner, the cardiovascular center responds via increased parasympathetic discharge in efferent motor fibers of the vagus nerves to the heart and by decreased sympathetic discharge in the cardiac accelerator nerves to the heart. The resulting decreases in heart rate and force of contraction lower cardiac output. In addition, the cardiovascular center sends out fewer sympathetic impulses along vasomotor fibers that normally cause vasoconstriction. The result is vasodilatation, which lowers systemic vascular resistance.

### Carotid Sinus Baroreceptors

It has been demonstrated that there are two functionally different carotid sinus baroreceptors, where each type may play a different role in the regulation of blood pressure. Reference is now made to FIG. 2A, which is a plot of baroreceptor activity, measured on the ordinate as pulses or spikes per second against carotid sinus pressure on the abscissa, measured in mm Hg. Type I baroreceptors are characterized by a discontinuous hyperbolic transduction curve 10. Specifically, the electrical discharge pattern of these baroreceptors is such that, until a threshold carotid sinus pressure has been achieved, no signal is produced. However, when a carotid sinus pressure reaches the threshold, type I baroreceptor discharge commences abruptly, with an initial firing rate of about 30 spikes per second. Saturation occurs at about 200 mm Hg, at which the firing rate saturates at about 50 spikes per second. The nerve fibers connected to these types of baroreceptors are mostly thick, myelinated type A-fibers. Their conduction velocity is high, and they start firing at a relatively low threshold current (i.e., they have high impedance). The above characteristics for the type I baroreceptors suggest that they are involved in the dynamic regulation of arterial blood pressure, regulating abrupt, non-tonic changes in blood pressure.

Type II baroreceptors are pressure transducers that are characterized by a continuous transduction curve 12. Specifically, the electrical discharge pattern of these baroreceptors is such that they transmit impulses even at very low levels of arterial blood pressure. Consequently, there is no defined threshold for type II baroreceptors. The typical firing rate of type II baroreceptors in a normotensive individual is about five spikes per second. At a carotid sinus pressure of about 200 mm Hg, the firing rate saturates at about 15 spikes per second. The nerve fibers connected to type II baroreceptors are either thin, myelinated type A fibers, or unmyelinated type C fibers. Their conduction velocity is low and, when stimulated experimentally, they start firing at a relatively high threshold current, due to their relatively low impedance. The above characteristics of type II baroreceptors suggest that they are involved in the tonic regulation of arterial blood pressure, and that they play a role in the establishment of baseline blood pressure (i.e., diastolic blood pressure).

### Modulation of Baroreceptor Activity

The baroreceptive endings of a carotid sinus nerve and the aortic depressor nerve are the peripheral terminals of a group of sensory neurons with their soma located in the petrosal and nodose ganglia. The endings terminate primarily in the tunica adventitia of a carotid sinus and aortic arch. When stretched, they depolarize. Action potentials are consequently triggered from a spike-initiating zone on the axon near the terminal. The action potentials travel centrally to the nucleus tractus solitarius in the medulla. There, the sensory neurons synapse with a second group of central neurons, which in turn transmit impulses to a third group of efferent neurons that control the parasympathetic and sympathetic effectors of the cardiovascular system. The vascular structure of a carotid sinus and aortic arch determines the deformation and strain of the baroreceptor endings during changes in arterial pressure. For this reason, structural changes in the large arteries and decreased vascular distensibility, also known as compliance, are often considered the predominant mechanisms responsible for decreased baroreflex sensitivity and resetting of baroreceptors, which occur in hypertension, atherosclerosis, and aging.

The process of mechanoelectrical transduction in the baroreceptors depends on two components: (1) a mechanical component, which is determined by the viscoelastic characteristics of coupling elements between the vessel wall and the nerve endings, and (2) a functional component, which is related to (a) ionic factors resulting from activation of channels or pumps in the neuronal membrane of the baroreceptor region, which alter current flow and cause depolarization resulting in the generation of action potentials, and (b) paracrine factors released from tissues and cells in proximity to the nerve endings during physiological or pathological states. These cells include endothelial cells, vascular muscle cells, monocytes, macrophages, and platelets. The paracrine factors include prostacyclin, nitric oxide, oxygen radicals, endothelin, platelet-derived factors, and other yet unknown compounds. Extensive animal studies conducted in the 1990s support the concept that the mechanoelectrical transduction in baroreceptor neurons occurs through stretch-activated ionic channels, whose transduction properties are affected by the aforementioned factors.

There exists evidence indicating a dependency of the baroreflex on the temporal characteristics of discharges in the cardiovascular afferent fibers. The coupling of afferent baroreceptor activity with the central group of neurons leads to inhibition of sympathetic nerve activity. This coupling was examined by determining the relationship between afferent baroreceptor activity and efferent sympathetic nerve activity measured simultaneously.

Sustained inhibition of sympathetic nerve activity is not simply a function of baroreceptor spike frequency, but depends on the phasic burst pattern, with on and off periods during systole and diastole, respectively. Sympathetic nerve activity is disinhibited, because of what may be viewed as a “central adaptation,” during nonpulsatile, nonphasic baroreceptor activity. It is not actually the pulse pressure that is important in sustaining sympathetic inhibition, but rather the magnitude of pulsatile distension of a carotid sinus and the corresponding phasic baroreceptor discharge. One would predict that a decrease in large artery compliance, as might occur in chronic hypertension or atherosclerosis, could result in a decrease in pulsatile distension of a carotid sinus and a blunting of the phasicity of baroreceptor input. There is progressive loss of the buffering capacity of the baroreflex because of central adaptation. It has been shown experimentally that the reflex inhibition of sympathetic nerve activity is most pronounced at lower frequencies of pulsatile pressure and during bursts of baroreceptor activity (between 1 and 2 Hz). When the burst or pulse frequency exceeded 3 Hz, there is known to be a significant disinhibition of sympathetic nerve activity, despite a maintained high level of total baroreceptor spike frequency per unit time. Thus, at very rapid pulse rates the efficiency of afferent-efferent coupling is reduced.

In a study conducted using young (1 year old) and old (10 years old) beagle dogs, it was found that the reflex inhibition of sympathetic nerve activity after a rise in carotid sinus pressure was maintained in the young but was very transient in the old dogs. The “escape” of sympathetic nerve activity from baroreflex inhibition occurred in the old dogs despite a maintained increase in afferent baroreceptor activity. Thus, the major defect in the baroreflex with aging may not be a structural vascular defect or an impaired baroreceptive process, but rather a central neural defect in the afferent-efferent coupling. It is proposed in U.S. Pat. No. 4,201,219 to employ a neurodetector device in order to generate pulsed electrical signals. The frequency of the impulses is utilized to pace the heart directly in order to modify the cardiac rate. This approach has not been generally accepted, as there were serious technical difficulties with the implantation, and the reliability of the apparatus. In U.S. Pat. No. 3,650,277 it is proposed to treat hypertension by stimulating afferent nerve paths from the baroreceptors of a patient, in particular the nerves from a carotid sinus. Short electrical pulses are used during a limited period of the cardiac cycle. It is necessary to synchronize an electrical signal generator to the heart activity of the patient, either by measuring electrical activity of the heart, or by using a transducer that is capable of measuring instantaneous blood pressure.

Another attempt at simulating the baroreceptor reflex is disclosed in U.S. Pat. No. 4,791,931, wherein a pressure transducer and a cardiac pacemaker are implanted. The pacing rate is variable and is responsive to arterial pressure.

### Peripheral Chemoreceptors and Central Chemoreceptors

The primarily function of chemoreceptors is to regulate respiratory activity. This is an important mechanism for maintaining arterial blood pO₂, pCO₂, and pH within appropriate physiological ranges. For example, a fall in arterial pO₂ (hypoxemia) or an increase in arterial pCO₂ (hypercapnia) leads to an increase in the rate and depth of respiration through activation of the chemoreceptor reflex. Chemoreceptor activity, however, also affects cardiovascular function either directly (by interacting with medullary vasomotor centers) or indirectly (via altered pulmonary stretch receptor activity). Respiratory arrest and circulatory shock (these conditions decrease arterial pO₂ and pH, and increase arterial pCO₂) dramatically increase chemoreceptor activity leading to enhanced sympathetic outflow to the heart and vasculature via activation of the vasomotor center in the medulla. Cerebral ischemia activates central chemoreceptors, which produces simultaneous activation of sympathetic and vagal nerves to the cardiovascular system.

The carotid bodies are located on the external carotid arteries near their bifurcation with the internal carotids. Each carotid body is a few millimeters in size and has the distinction of having the highest blood flow per tissue weight of any organ in the body. Afferent nerve fibers join with the sinus nerve before entering the glossopharyngeal nerve. A decrease in carotid body blood flow results in cellular hypoxia, hypercapnia, and decreased pH that lead to an increase in receptor firing. The threshold pO2 for activation is about 80 mmHg (normal arterial pO₂ is about 95 mmHg). Any elevation of pCO₂ above a normal value of 40 mmHg, or a decrease in pH below 7.4 causes receptor firing. If respiratory activity is not allowed to change during chemoreceptor stimulation (thus removing the influence of lung mechanoreceptors), then chemoreceptor activation causes bradycardia and coronary, and said central (both via vagal activation) and systemic vasoconstriction (via sympathetic activation). If respiratory activity increases, then sympathetic activity stimulates both the heart and vasculature to increase arterial pressure.

### Aneurysmal Subarachnoid Hemorrhage

Aneurysmal Subarachnoid Hemorrhage (SAH) is a condition in which bleeding occurs in the subarachnoid space due to a ruptured aneurysm. SAH is a life-threatening disease that accounts for approximately 5% of all strokes; it is estimated to affect 30,000 Americans annually. The risk for SAH is increased in individual who smoke, drink alcohol in excess and in hypertensive individuals. Early repair of the recently ruptured aneurysm is an imperative part of caring for SAH patients. Therefore, admittance to a neurologic-neurosurgical intensive care unit and aggressive care may prevent further deterioration that can substantially affect patient outcome.

SAH is commonly graded by using the World Federation on Neurological Societies (WFNS) grading scale, which is based on the Glasgow Coma Scale. Patients who survive the initial hours after the hemorrhage and have their aneurysms secured by clipping, coiling or stenting are still at risk for severe complications, especially within the first 2 weeks after the hemorrhage. One of the most severe complications is delayed cerebral ischemia caused by symptomatic vasospasm, which occurs mostly between days 4 and 10 after SAH. As a result of the symptomatic vasospasm many die or suffer permanent morbidity and it has been described as the single most important cause of morbidity and mortality in patients whose ruptured aneurysm is successfully treated.

Post SAH vasospasm incidence is in between 30% to 70% which 50% of those patients experiencing neurologic complications.

### Current Cerebral Vasospasm Treatment

According to Bederson et al, (Guidelines for the Management of Aneurysmal Subarachnoid Hemorrhage A Statement for Healthcare Professionals From a Special Writing Group of the Stroke Council, American Heart Association. Stroke 2009, 40:994-1025: originally published online Jan. 22, 2009) the following are the four recommendations for management of cerebral vasospasm:

1. Oral Nimodipine is indicated to reduce poor outcome related to aneurysmal SAH. The value of other calcium antagonists, whether administered orally or intravenously, remains uncertain.

2. Treatment of cerebral vasospasm begins with early management of the ruptured aneurysm, and in most cases, maintaining normal circulating blood volume and avoiding hypovolemia are probably indicated.

3. One reasonable approach to symptomatic cerebral vasospasm is volume expansion, induction of hypertension, and hemodilution (triple-H therapy).

4. Alternatively, cerebral angioplasty and/or selective intra-arterial vasodilator therapy may be reasonable after, together with, or in place of triple-H therapy, depending on the clinical scenario.

Nimodipine, a calcium channel blocker administered orally, improves overall patient outcome after SAH. The drug does not increase the caliber of narrowed cerebral arteries on cerebral angiography. Rather, the calcium channel blockade seems to have a neuroprotective effect. For patients who become symptomatic with delayed ischemic deficit due to vasospasm, more aggressive intravascular volume expansion and induced hypertension are used.

If medical therapy for symptomatic vasospasm has been maximized and neurologic symptoms prove refractory, endovascular therapies can be considered. Intra-arterial papaverine infusion acts immediately and increases arterial diameter and cerebral blood flow, but its effects are short-lived. Balloon angioplasty is particularly effective as a durable means of alleviating arterial narrowing and preventing stroke in patients with sympatomatic vasospam after aneurysmal SAH, however, the procedure is, technically complicated, limited in small vessel pathology and involved with significant risks.

A systematic review of 14 trials with a combined number of 4,235 patients found that despite a decreased incidence of radiographic vasospasm, pharmaceutical treatment after SAH did not improve clinical outcome7.

A safe and effective minimally invasive therapeutic tool is an unmet need for reversal cerebral vasospasm and minimizing neurological damage.

### SUMMARY OF THE INVENTION

It is an object of the present invention to provide a medical apparatus and a method for the treatment of physiological disorders such as, but not limited to cerebral brain vaso spasm, ischemia and brain injury. More particularly this invention relates to the stimulation of at least one selected from a group consisting of carotid sinus nerve, aortic nerve, chemoreceptors adjacent to the bifurcation of the carotid, baroreceptors adjacent to the bifurcation of the carotid, aortic arch chemoreceptors and aortic arch baroreceptors in order induce vasodilatation in blood vessels of the brain.

It is one object of the present invention to provide a system for treating a medical condition in a living body of a patient, comprising: a delivery system comprising: (a) at least one electro stimulation module implant, optionally intended to be reversibly implanted in a patient; the implant comprising a proximal end and a distal end; the distal end comprising at least one distal end member with at least one electrically conductive electrode mounted in said distal end member and wiring connecting the electrode to the proximal end and (b) an electrical stimulation unit, intended for producing and controlling an electrical waveform, the waveform to be delivered to the electrostimulation module implant via electrical connectors, either by wiried or by wireless means wherein the electrostimulation module implant is implanted adjacent to at least one of a group consisting of: the carotid sinus nerve, the aortic nerve, the common carotid artery, the external carotid artery, internal carotid artery, carotid artery bifurcation, carotid body, aortic body and aortic arch receptors within said living body; further wherein said electrical stimulation unit is maintained outside said patient and is adapted to program and control said electrical stimulation signal such that said electrical waveform is delivered by means of wiring or wireless communication between implant and electrical stimulation unit.

The electric stimulation can be optimized in order to achieve a well-focused and effective nerve stimulation. Several parameters can be adjusted to achieve this. These parameters are designed to control the shape and strength of the electrical field and its anatomic location.

Some of these parameters are:
Current control or voltage control of the electrical regime.
Under current control conditions, the current applied to the electrodes is typically in the range of 0-10 mA, but current is not limited to this range.
Under voltage control conditions, the voltage applied to the electrodes is typically in the range of 0-25V, but voltage is not limited to this range.
The signal symmetry can be monophasic or biphasic.
The distance between the effective electrodes can be in the range of about 1 mm to 20 mm, but the distance is not limited to this range.
The number of electrodes: there is a plurality of electrodes.
The electrodes can be activated in any combination and in any order; this combinations and the order can be changed during a stimulation session, either as part of a pre-determined sequence or in response to feedback from the patient.
Size of the electrodes: Electrodes can range from about a tenth of a millimeter long to about 10 millimeter long.
Shape of the electrodes: electrodes can be cylindrical, partly-cylindrical with the base forming a sector of a circle, spherical, hemispheric, forming a section of a sphere, cylindrical with a polygonal base, cylindrical with a base forming a sector of a polygon, in the form of a triangular prism, in the form of a rectangular solid, in the form of an octahedral solid, in the form of a dodecahedral solid, in the form of an icosahedral solid, rectangular prism, ellipsoid, parallelepiped, star-shaped solid, helical and any combination thereof. Electrodes can be mounted longitudinally, transversely, or at an angle to the supports.
Positioning of electrodes—longitudinally, across, beside or any combination that will electrically cover to the desired anatomic location
Signal Profile—burst, prolonged, intermittent and any combination thereof. Individual groups of signals, such as but not limited to individual bursts, can have a step profile, a ramped profile that increases monotonically from the beginning to the end of the group of signals, a ramped profile that decreases monotonically from the beginning to the end of the group, a ramped profile which increases from a small value to a predetermined value, then remains constant until the end of the group, a ramped profile that starts at a predetermined value, remains at that value for a predetermined portion of the group, then decreases to a small value at the end of the group, a sinusoidal signal profile, a triangular signal profile, and any combination thereof.

It is an object of the present invention to provide a system for treating a medical condition in a living body of a patient, comprising (a) at least one implant, adapted to be retrievably implanted in the patient; the implant comprising: at least one electrostimulation module comprising a proximal end and a distal end, the distal end comprising at least one first distal end member; and at least one electrically conductive electrode mounted in the at least one first distal end member; and (b) at least one electrical stimulation unit, adapted for producing an electrical waveform and connected to at least one of the electrodes wherein the implant is implanted adjacent to at least one of the group consisting of: the carotid sinus nerve, the aortic nerve, the common carotid artery, the external carotid artery, internal carotid artery, carotid artery bifurcation, carotid body, aortic body, aortic arch receptors and any combination thereof within the living body; further wherein the electrical stimulation unit is maintained outside the patient's body and is adapted to program, generate, control and deliver the electrical waveform, the delivery to the electrodes being via at least one of a group consisting of a wired connection and a wireless connection.

The system according to claim 1, wherein the electrode of the implant is positioned such that an electrical excitatory waveform generated by the electrical stimulation unit is adapted to at least concurrently increase cerebral perfusion in a region in the subject's brain by more than about 7%, while changing mean arterial blood pressure by less than about 10%.

The system according to claim 1, wherein the electrode of the implant is positioned such that an electrical excitatory waveform generated by the electrical stimulation unit is adapted to at least concurrently increase cerebral perfusion in a region in the subject's brain by more than about 12%, while changing mean arterial blood pressure by less than about 7%.

The system according to claim 1, wherein the electrode of the implant is positioned such that an electrical excitatory waveform generated by the electrical stimulation unit is adapted to at least concurrently increase cerebral perfusion in a region in the subject's brain by more than about two times the percentage increase in mean arterial blood pressure.

The system according to claim 1, wherein the electrode of the implant is positioned such that an electrical excitatory waveform generated by the electrical stimulation unit is adapted to at least concurrently increase cerebral perfusion in a region in the subject's brain by more than about four times the percentage increase in mean arterial blood pressure.

It is an object of the present invention to provide the system as defined above, wherein the implant is configured to be implanted by at least one means selected from a group consisting of endovascular means, extravascular percutaneous means and extravascular surgical means.

It is an object of the present invention to provide the system as defined above, wherein the endovascular means comprises a delivery catheter configured to deliver, position and retrieve the implant.

It is an object of the present invention to provide the system as defined above, wherein the delivery catheter is inserted through an insertion sheath.

It is an object of the present invention to provide the system as defined above, wherein the delivery catheter is inserted through a guiding catheter.

It is an object of the present invention to provide the system as defined above, wherein the electrical stimulation unit is configured to be externally disposed on the body of the patient.

It is an object of the present invention to provide the system as defined above, wherein the electrical stimulation unit is configured and shaped as at least one selected from the group consisting of: a belt, necklace, collar, bracelet, armlet, anklet, ring and any combination thereof.

It is an object of the present invention to provide the system as defined above, wherein the electrical stimulation unit is located around the neck of the patient.

It is an object of the present invention to provide the system as defined above, wherein the electrical stimulation unit comprises at least one antenna.

It is an object of the present invention to provide the system as defined above, wherein the implant additionally comprises at least one transmitter and receiver.

It is an object of the present invention to provide the system as defined above, wherein the transmitter is adapted to transmit feedback signals to the electrical stimulation unit.

It is an object of the present invention to provide the system as defined above, wherein at least one of the first distal end members is at a position selected from a group consisting of: within, proximate to and any combination thereof, the position relative to at least one of a group consisting of: the carotid sinus nerve, the common carotid artery, the external carotid artery, the internal carotid artery, the carotid artery bifurcation, the carotid body, and any combination thereof, so as to enable the stimulation by the electrode of at least one selected from a group consisting of: carotid sinus nerve, chemoreceptors in the arteries adjacent to the bifurcation of the carotid, baroreceptors in the arteries adjacent to the bifurcation of the carotid, and any combination thereof.

It is an object of the present invention to provide the system as defined above, wherein the implant additionally comprises at least one of a third distal end member positioned adjacent to at least one selected from a group consisting of: the aortic nerve, the aortic body and the aortic arch receptors, so as to enable the stimulation by the electrode of at least one selected from the group consisting of: the aortic nerve, aortic arch chemoreceptors in the aortic arch receptors, aortic arch baroreceptors in the aortic arch receptors, the aortic body and any combination thereof.

It is an object of the present invention to provide the system as defined above, wherein the electric field generated by the electrodes is optimized by at least one optimization parameter selected from a group consisting of: electrical characteristic of the electrodes, electrically effective shape of the electrodes, location of the electrodes relative to each other, electrical waveform characteristic and any combination thereof; so as to control and optimize the location and magnitude of the electric field and the rate at which energy is transferred to the electric field so as to enable the effective stimulation so as to enable the stimulation by the electrode of at least one selected from a group consisting of: carotid sinus nerve, aortic nerve, chemoreceptors adjacent to the bifurcation of the carotid, baroreceptors adjacent to the bifurcation of the carotid, aortic arch chemoreceptors, aortic arch baroreceptors and any combination thereof.

It is an object of the present invention to provide the system as defined above, wherein the electrical waveform characteristic of the electrodes comprises at least one selected from a group consisting of: (a) current being in the range of about 0 to about 10 mA; (b) voltage in the range of about 0 to about 25 V; (c) signal shape selected from a group consisting of rectangular, triangular, sinusoidal & any combination thereof; (d) signal profile selected from a group consisting of monophasic, biphasic and any combination thereof; (e) pulse width or duration range of about 0.1 msec to about 4 msec; (f) pulse repetition or frequency range of about 5 Hz to about 100 Hz (g) signal regime selected from a group consisting of: burst, prolonged, intermittent and any combination thereof.

It is an object of the present invention to provide the system as defined above, wherein the electric field generated by the at least two electrodes is optimized so as to enhance the responsiveness of at least one selected from a group consisting of: carotid sinus nerve, aortic nerve, chemoreceptors adjacent to the bifurcation of the carotid, baroreceptors adjacent to the bifurcation of the carotid, aortic arch chemoreceptors, aortic arch baroreceptors and any combination thereof.

It is an object of the present invention to provide the system as defined above, wherein the electric field generated by the at least two electrodes is optimized so as to enhance at least one selected from a group consisting of dilation of blood vessels in the brain, cerebral blood flow, cerebral perfusion and any combination thereof.

It is an object of the present invention to provide the system as defined above, wherein the system further comprises at least one anchoring member; the anchoring member is characterized by at least two configurations; a collapsed state adapted to allow free longitudinal motion of the at least one electrode along the insertion path to the implantation site, and an expanded state, in which the anchoring member is in contact with at least one of a group consisting of: a blood vessel wall, surrounding tissue in the neck exterior to a blood vessel proximate to the carotid artery bifurcation.

It is an object of the present invention to provide the system as defined above, wherein the at least one anchoring member is reversibly transitionable between the collapsed state and the expanded state.

It is an object of the present invention to provide the system as defined above, wherein the anchoring member additionally comprises at least one selected from a group consisting of an RF-opaque portion and an echogenic portion.

It is an object of the present invention to provide the system as defined above, wherein the anchoring member is selected from a group consisting of: a basket, cage, mesh, stent, balloon, clamp and any combination thereof.

It is an object of the present invention to provide the system as defined above, wherein the transition from collapsed state to expanded state is effected by at least one of a group consisting of: a magnetic field acting on one or more parts of the anchoring member, an electric field acting on one or more parts of the anchoring member, fluid distending all or part of the anchoring member, wires or other mechanical connectors acting on one or more parts of the anchoring member, removal of an external sheath, removal of an external covering, and any combination thereof.

It is an object of the present invention to provide the system as defined above, wherein the mesh is one of a group consisting of: woven, knitted, auxetic and any combination thereof.

It is an object of the present invention to provide the system as defined above, wherein the stent is one of a group consisting of: woven, knitted, auxetic and any combination thereof.

It is an object of the present invention to provide the system as defined above, wherein the anchoring member is coupled to at least one of a group consisting of the distal end, the proximal end, and the central portion of the electrostimulation module.

It is an object of the present invention to provide the system as defined above, wherein the anchoring member comprises spring-like members, such that the transition from collapsed state to expanded state is effected by removal of a sheath-like covering and transition from expanded state to collapsed state is effected by replacement of the sheath-like covering.

It is an object of the present invention to provide the system as defined above, wherein the sheath-like covering is a tube.

It is an object of the present invention to provide the system as defined above, wherein the transition from the collapsed state to the expanded state of the anchoring member is obtained by linear reciprocal movement of the distal end towards and away from the proximal end.

It is an object of the present invention to provide the system as defined above, wherein the electrode being characterized by a generally circular cross section

It is an object of the present invention to provide the system as defined above, wherein the electrode comprises at least one material selected from a group consisting of: stainless steel, Platinum alloy, Iridium alloy, Silver alloy, Silver Chloride alloy, Nickel Titanium alloy, gold alloy and any combination thereof.

It is an object of the present invention to provide the system as defined above, further comprising means for estimating at least one of a group consisting of: cerebral blood flow, cerebral perfusion, cerebral blood oxygen saturation, intra cranial pressure and arterial blood pressure, the means are adapted to generate at least one control signal indicative of the at least one of the group, and wherein the electrical stimulation unit is capable of adapting the electrical waveform in accordance with the control signal so as to control a parameter of the at least one of the group.

It is an object of the present invention to provide the system as defined above, wherein data to enable the estimation by the means for estimating are received from at least one selected from a group consisting of a source external to the system, a source internal to the system and any combination thereof.

It is an object of the present invention to provide the system as defined above, wherein the control signal comprises a desired duration of treatment.

It is an object of the present invention to provide the system as defined above, wherein the control signal comprises a desired intensity of treatment.

It is an object of the present invention to provide the system as defined above, wherein the control signal comprises a time-dependent control over a parameter of the at least one of the group.

It is an object of the present invention to provide the system as defined above, wherein the means for estimating cerebral blood flow comprises at least one selected from a group consisting of transcranial Doppler flowmeter (TCD), computerized tomography (CT), CT angiography (CTA), magnetic resonance imaging (MRI), positron emission tomography (PET), single photon emission computerized tomography (SPECT), laser Doppler flowmeter, Doppler enhanced ultrasound, UTLight□ technology, nICP monitor and any combination thereof.

It is an object of the present invention to provide the system as defined above, wherein the electric field generated by the electrodes comprises an electrical waveform characterized by a pulse train; further wherein the pulse train comprises intermittently active and inactive periods, the active periods characterized by a substantially non-zero electrical energy being contained in the waveform, the inactive period comprises a substantially zero electrical energy being contained in the waveform.

It is an object of the present invention to provide the system as defined above, wherein the pulse comprises at least one of a group consisting of: a biphasic pulse and a monophasic pulse.

It is an object of the present invention to provide the system as defined above, wherein a pulse repetition rate is between about 5 pulses per second and about 100 pulses per second.

It is an object of the present invention to provide the system as defined above, wherein an electrical waveform is driven to the chemoreceptor(s) nerves(s) and optionally to the baroreceptor(s) nerve(s), wherein the step occurs in a mutually exclusive manner, so that when the electrical waveform is driven to the chemoreceptor nerve, electrical waveform is not driven to the other chemoreceptor nerve or baroreceptor(s) nerve(s), and vice versa, so as to reduce physiological tolerance (i.e. tachyphylaxis) of the cerebral vasodilatation to the electrical waveform.

It is an object of the present invention to provide the system as defined above, wherein an electrical waveform is driven to the chemoreceptor(s) nerve(s) and optionally to the baroreceptor(s) nerve(s), wherein the step occurs in a partially simultaneous manner, so that during a first phase of treatment, the electrical waveform is driven to more than a single chemoreceptor nerve and optionally baroreceptor nerve, during a second phase of treatment, the electrical waveform is driven only to a single chemoreceptor or optionally to baroreceptor nerve, during a third phase of treatment, the electrical waveform is driven only to another a single chemoreceptor or optionally to baroreceptor nerve, and during a fourth phase of treatment no electrical waveform is driven to either chemoreceptor nerve or to baroreceptor nerve, and wherein the first, second, third, and fourth phases of treatment are intermittently occurring.

It is an object of the present invention to provide the system as defined above, The system according to claim 1, wherein an electrical waveform is driven to the chemoreceptor(s), wherein the step occurs in a partially simultaneous manner, so that during a first phase of treatment, the electrical waveform is driven only to a single chemoreceptor nerve, and during a second phase of treatment the electrical waveform is driven to both chemoreceptor nerves, during a third phase of treatment, the electrical waveform is driven only to another single chemoreceptor nerve, and during a fourth phase of treatment the electrical waveform is driven to more than a single chemoreceptor nerve, and wherein the first, second, third, and fourth phases of treatment are intermittently occurring.

It is an object of the present invention to provide the system as defined above, further comprising means adapted to perform measurement of a physiological parameter in the subject, and adjusting the electrical waveform accordingly, wherein the physiological parameter is selected from the group consisting of: blood pressure, blood flow, blood velocity, cerebral perfusion, intra cranial pressure, cerebral oxygen saturation, metabolic state of cerebral tissue, metabolic state of brain, heart rate, respiratory rate and any combination thereof.

It is an object of the present invention to provide the system as defined above, wherein the medical condition is selected from the group consisting of: cerebral hemorrhage, subarachnoid hemorrhage, cerebral vasospasm, brain ischemia, ischemic stroke, delayed cerebral ischemia, traumatic brain injury, Vascular Dementia (VaD) also known as Multi-Infarct Dementia, aphasia, migraine, chronic headaches, cluster headache and any combination thereof.

It is an object of the present invention to provide a method for treating a medical condition in a living body of a patient, comprising the following steps: (a) identifying a subject having a predetermined medical condition; (b) providing at least one delivery system comprising (i) at least one implant, adapted to be retrievably implanted in the patient; the implant comprising at least one electrostimulation module comprising a proximal end and a distal end, the distal end comprising at least one first distal end member; and at least one electrically conductive electrode mounted in the at least one first distal end member; and (ii) at least one electrical stimulation unit, adapted for producing an electrical waveform and connected to at least one of the electrodes; (c) implanting the implant by positioning at least one of the first distal end member, the position selected from a group consisting of: within or proximate to, the position relative to at least one selected from a group consisting of: the carotid sinus nerve, the common carotid artery, the external carotid artery, the internal carotid artery, the carotid artery bifurcation, the carotid body and any combination thereof; (d) positioning the electrical stimulation unit outside the patient; (e) activating an electrical waveform by means of the electrical stimulation unit; and (f) driving the electrical waveform from the electrical stimulation unit to the at least one electrode via at least one of the first distal end member; thereby stimulating at least one selected from a group consisting of: carotid sinus nerve, chemoreceptors in the arteries adjacent to the bifurcation of the carotid, baroreceptors in the arteries adjacent to the bifurcation of the carotid, and any combination thereof; wherein the implant is implanted adjacent to least one of the group consisting of: a carotid body, the carotid sinus nerve, the aortic nerve, the common carotid artery, the external carotid artery, the internal carotid artery, the carotid artery bifurcation, the aortic body, the aortic arch receptors within the living body; further wherein the electrical stimulation unit is maintained outside the patient's body and is adapted to program, generate, control and deliver the electrical waveform, the delivery to the electrodes being via at least one of a group consisting of a wired connection and a wireless connection.

It is an object of the present invention to provide the method as defined above, wherein the electrode of the implant is positioned such that an electrical excitatory waveform generated by the electrical stimulation unit is adapted to at least concurrently increase at least one of a group consisting of: cerebral perfusion and cerebral blood flow in a region in the subject's brain by more than about 7%, while changing mean arterial blood pressure by less than about 10%.

It is an object of the present invention to provide the method as defined above, wherein the electrode of the implant is positioned such that an electrical excitatory waveform generated by the electrical stimulation unit is adapted to at least concurrently increase increase at least one of a group consisting of cerebral perfusion and cerebral blood flow in a region in the subject's brain by more than about 12%, while changing mean arterial blood pressure by less than about 7%.

It is an object of the present invention to provide the method as defined above, wherein the electrode of the implant is positioned such that an electrical excitatory waveform generated by the electrical stimulation unit is adapted to at least concurrently increase increase at least one of a group consisting of: cerebral perfusion and cerebral blood flow in a region in the subject's brain by more than about two times the percentage increase in mean arterial blood pressure.

It is an object of the present invention to provide the method as defined above, wherein the electrode of the implant is positioned such that an electrical excitatory waveform generated by the electrical stimulation unit is adapted to at least concurrently increase increase at least one of a group consisting of: cerebral perfusion and cerebral blood flow in a region in the subject's brain by more than four times the percentage increase in mean arterial blood pressure.

It is an object of the present invention to provide the method as defined above, wherein the implant is configured to be implanted by at least one means selected from a group consisting of: endovascular means, extravascular percutaneous means and extravascular surgical means.

It is an object of the present invention to provide the method as defined above, wherein the endovascular means comprises a delivery catheter configured to deliver, position and retrieve the implant.

It is an object of the present invention to provide the method as defined above, wherein the delivery catheter is inserted through insertion sheath.

It is an object of the present invention to provide the method as defined above, wherein the delivery catheter is inserted through a guiding catheter.

It is an object of the present invention to provide the method as defined above, wherein the electrical stimulation unit is configured to be externally disposed on the body of the patient.

It is an object of the present invention to provide the method as defined above, wherein the electrical stimulation unit is configured and shaped as at least one selected from a group consisting of: a belt, necklace, collar, bracelet, armlet, anklet, ring and any combination thereof.

It is an object of the present invention to provide the method as defined above, wherein the electrical stimulation unit is located around the neck of the patient.

It is an object of the present invention to provide the method as defined above, wherein the electrical stimulation unit comprises at least one antenna.

It is an object of the present invention to provide the method as defined above, wherein the implant additionally comprises at least one transmitter and receiver.

It is an object of the present invention to provide the method as defined above, additionally comprising step of transmitting at least one feedback signal to the electrical stimulation unit.

It is an object of the present invention to provide the method as defined above, wherein the implant additionally comprises at least one of a third distal end member positioned adjacent to at least one selected from a group consisting of: aortic nerve, aortic body and aortic arch receptors, so as to enable the stimulation by the electrode of at least one selected from a group consisting of: the aortic nerve, aortic arch chemoreceptors in the aortic arch receptors, aortic arch baroreceptors in said aortic arch receptors, the aortic body and any combination thereof.

It is an object of the present invention to provide the method as defined above, wherein the system is configured such that the electric field generated by the electrodes is optimized by at least one optimization parameter selected from a group consisting of electrical characteristic of the electrodes, electrically effective shape of the electrodes, location of the electrodes relative to each other, electrical waveform characteristic and any combination thereof; so as to control and optimize the location and magnitude of said electric field and the rate at which energy is transferred to said electric field so as to enable the effective stimulation by said electrode of at least one selected from a group consisting of: carotid sinus nerve, aortic nerve, chemoreceptors adjacent to the bifurcation of the carotid, baroreceptors adjacent to the bifurcation of the carotid, aortic arch chemoreceptors, aortic arch baroreceptors and any combination thereof.

It is an object of the present invention to provide the method as defined above, wherein the electrical characteristic of the electrodes comprises at least one selected from a group consisting of (a) current being in the range of about 0 to about 10 mA; (b) voltage in the range of about 0 to about 25 V; (c) signal shape selected from a group consisting of rectangular, triangular, sinusoidal & any combination thereof; (d) signal profile selected from a group consisting of monophasic, biphasic and any combination thereof; (e) pulse width or duration range of about 0.1 msec to about 4 msec; (f) pulse repetition or frequency range of about 5 Hz to about 100 Hz (g) signal regime selected from a group consisting of: burst, prolonged, intermittent and any combination thereof.

It is an object of the present invention to provide the method as defined above, wherein the electric field generated by the at least two electrodes is optimized so as to enhance the responsiveness of at least one selected from a group consisting of: carotid sinus nerve, aortic nerve, chemoreceptors adjacent to the bifurcation of the carotid, baroreceptors adjacent to the bifurcation of the carotid, aortic arch chemoreceptors, aortic arch baroreceptors and any combination thereof.

It is an object of the present invention to provide the method as defined above, wherein the electric field generated by the at least two electrodes is optimized so as to enhance at least one selected from a group consisting of: dilation of blood vessels in the brain, cerebral blood flow, cerebral perfusion and any combination thereof.

It is an object of the present invention to provide the method as defined above, wherein the system further comprises at least one anchoring member; the anchoring member is characterized by at least two configurations; a collapsed state adapted to allow free longitudinal motion of the at least one electrode along the insertion path to the implantation site, and an expanded state, in which the anchoring member is in contact with at least one of a group consisting of: a blood vessel lumen, surrounding tissue in the neck exterior to a blood vessel proximate to the carotid artery bifurcation.

It is an object of the present invention to provide the method as defined above, wherein the at least one endovascular anchoring member is reversibly transitionable between the collapsed state and the expanded state.

It is an object of the present invention to provide the method as defined above, wherein the anchoring member additionally comprises at least one of a group consisting of an RF-opaque portion and an echogenic portion.

It is an object of the present invention to provide the method as defined above, wherein the anchoring member is selected from a group consisting of: a basket, cage, mesh, stent, balloon, clamp and any combination thereof.

It is an object of the present invention to provide the method as defined above, wherein the transition from collapsed state to expanded state is effected by at least one of a group consisting of: a magnetic field acting on one or more parts of the endovascular anchoring member, an electric field acting on one or more parts of the endovascular anchoring member, fluid distending all or part of the endovascular anchoring mechanism, wires or other mechanical connectors acting on one or more parts of the endovascular anchoring mechanism, removal of an external sheath, removal of an external covering, or any combination thereof.

It is an object of the present invention to provide the method as defined above, wherein the mesh is one of a group consisting of: woven, knitted, auxetic and any combination thereof.

It is an object of the present invention to provide the method as defined above, wherein the stent is one of a group consisting of: woven, knitted, auxetic and any combination thereof.

It is an object of the present invention to provide the method as defined above, wherein the anchoring member is coupled to at least one of a group consisting of: the distal end, the proximal end, and the central portion of the electrostimulation module.

It is an object of the present invention to provide the method as defined above, wherein the anchoring member comprises spring-like members, such that the transition from collapsed state to expanded state is effected by removal of a sheath-like covering and transition from expanded state to collapsed state is effected by replacement of the sheath-like covering.

It is an object of the present invention to provide the method as defined above, wherein the sheath-like covering is a tube.

It is an object of the present invention to provide the method as defined above, wherein the transition from the collapsed state to the expanded state of the anchoring member is obtained by linear reciprocal movement of the distal end towards and away from the proximal end.

It is an object of the present invention to provide the method as defined above, wherein the electrode being characterized by a generally circular cross section.

It is an object of the present invention to provide the method as defined above, wherein the electrode comprises at least one material selected from a group consisting of: stainless steel, Platinum alloy, Iridium alloy, Silver alloy, Silver Chloride alloy, Nickel Titanium alloy, Gold alloy or any combination thereof.

It is an object of the present invention to provide the method as defined above, further comprising means for estimating at least one of a group consisting of: cerebral blood flow, cerebral perfusion, cerebral blood oxygen saturation, intra cranial pressure and arterial blood pressure, the means are adapted to generate at least one control signal indicative of the at least one of the group, and wherein the electrical stimulation unit is capable of adapting the electrical waveform in accordance with the control signal so as to control a parameter of the at least one of the group.

It is an object of the present invention to provide the method as defined above, wherein data to enable the estimation by the means for estimating are received from at least one selected from a group consisting of a source external to the system, a source internal to the system and any combination thereof.

It is an object of the present invention to provide the method as defined above, wherein the control signal comprises a desired duration of treatment.

It is an object of the present invention to provide the method as defined above, wherein the control signal comprises a desired intensity of treatment.

It is an object of the present invention to provide the method as defined above, wherein the control signal comprises a time-dependent control over a parameter of the at least one of the group.

It is an object of the present invention to provide the method as defined above, wherein the means for estimating cerebral blood flow comprises at least one selected from a group consisting of: transcranial Doppler flowmeter, computerized tomography, CT angiography, magnetic resonance imaging, positron emission tomography, single photon emission computerized tomography, laser Doppler flowmeter, Doppler enhanced ultrasound, UTLight□ technology, nICP monitor and any combination thereof.

It is an object of the present invention to provide the method as defined above, further comprising means adapted to perform measurement of a physiological parameter in the subject, and adjusting the electrical waveform accordingly, wherein the physiological parameter is selected from the group consisting of: blood pressure, blood flow, blood velocity, cerebral perfusion, intra cranial pressure, cerebral oxygen saturation, metabolic state of cerebral tissue, metabolic state of brain, heart rate, respiratory rate and any combination thereof.

It is an object of the present invention to provide the method as defined above, wherein the electric field generated by the electrodes comprises an electrical waveform characterized by a pulse train; further wherein the pulse train comprises intermittently active and inactive periods, the active periods are characterized by a substantially non-zero electrical energy being contained in the waveform, the inactive period comprises a substantially zero electrical energy being contained in the waveform.

It is an object of the present invention to provide the method as defined above, wherein the pulse comprises at least one of a group consisting of a biphasic pulse and a monophasic pulse.

It is an object of the present invention to provide the method as defined above wherein a pulse repetition rate is between about 5 pulses per second and about 100 pulses per second.

It is an object of the present invention to provide the method as defined above, wherein an electrical waveform is driven to the chemoreeeptor(s) nerves(s) and optionally to the baroreceptor(s) nerve(s), wherein the step occurs in a mutually exclusive manner, so that when the electrical waveform is driven to the chemoreceptor nerve, electrical waveform is not driven to the other chemoreceptor nerve or baroreceptor(s) nerve(s), and vice versa, so as to reduce physiological tolerance (i.e. tachyphylaxis) of the cerebral vasodilatation to the electrical waveform.

It is an object of the present invention to provide the method as defined above, wherein an electrical waveform is driven to the chemoreceptor(s) nerve(s) and optionally to the baroreceptor(s) nerve(s), wherein the step occurs in a partially simultaneous manner, so that during a first phase of treatment, the electrical waveform is driven to more than a single chemoreceptor nerve and optionally baroreceptor nerve, during a second phase of treatment, the electrical waveform is driven only to a single chemoreceptor or optionally to baroreceptor nerve, during a third phase of treatment, the electrical waveform is driven only to another a single chemoreceptor or optionally to baroreceptor nerve, and during a fourth phase of treatment no electrical waveform is driven to either chemoreceptor nerve or to baroreceptor nerve, and wherein the first, second, third, and fourth phases of treatment are intermittently occurring.

It is an object of the present invention to provide the method as defined above, The system according to claim 1, wherein an electrical waveform is driven to the chemoreceptor(s), wherein the step occurs in a partially simultaneous manner, so that during a first phase of treatment, the electrical waveform is driven only to a single chemoreceptor nerve, and during a second phase of treatment the electrical waveform is driven to both chemoreceptor nerves, during a third phase of treatment, the electrical waveform is driven only to another single chemoreceptor nerve, and during a fourth phase of treatment the electrical waveform is driven to more than a single chemoreceptor nerve, and wherein the first, second, third, and fourth phases of treatment are intermittently occurring.

It is an object of the present invention to provide the method as defined above, further comprising means adapted to perform measurement of a physiological parameter in the subject, and adjusting the electrical waveform accordingly, wherein the physiological parameter is selected from a group consisting of: blood pressure, blood flow, blood velocity, cerebral perfusion, and metabolic state of brain.

It is an object of the present invention to provide the method as defined above, further comprising means adapted to perform measurement of physiological parameters in the subject, and adjusting the electrical waveform accordingly, wherein the physiological parameters are selected from the group consisting of: cerebral perfusion, blood pressure, blood flow, blood velocity, metabolic state of cerebral tissue, metabolic state of brain, heart rate, respiratory rate and any combination thereof.

It is an object of the present invention to provide the method as defined above, wherein the step of performing measurement comprises performing at least one selected from a group consisting of: continuous measurement, periodic measurement, intermittently continuous measurement and any combination thereof.

It is an object of the present invention to provide the method as defined above, wherein the condition is selected from the group consisting of: cerebral hemorrhage, subarachnoid hemorrhage, cerebral vasospasm, brain ischemia, ischemic stroke, delayed cerebral ischemia, traumatic brain injury, Vascular Dementia (VaD) also known as Multi-Infarct Dementia, aphasia, migraine, chronic headaches, cluster headache and any combination thereof.

### BRIEF DESCRIPTION OF THE DRAWINGS

For a better understanding of these and other objects of the present invention, reference is made to the detailed description of the invention, by way of non-limiting example only, which is to be read in conjunction with the following drawings of which detailed description is presented further below, wherein:

FIGS. 1a-1c is a general anatomic description, schematically depicting the major vascular structures of the right throat, neck and head, up to the temple region;

FIGS. 2a-2f depicts electrical discharge patterns from baroreceptor and chemoreceptor fibers;

FIGS. 3-11 schematically illustrate different embodiments of the present invention;

FIG. 12 schematically illustrates an extravascular approach;

FIGS. 13-17 depict intermittent stimulation regimens for the baroreceptors and chemoreceptors to be effected according to several embodiments of the system of the present invention;

FIG. 18 illustrates an embodiment of the system;

FIG. 19 illustrates operation of an embodiment of the system;

FIG. 20 schematically illustrates vasodilation in the major cerebral arteries of swine during electrical stimulation of the chemoreceptors;

FIG. 21 depicts the locations of the electrodes within the cranial arteries; and

FIGS. 22-23 schematically illustrates the increase in cerebral perfusion in the major cerebral arteries of swine during electrical stimulation of the chemoreceptors.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide an efficient system and method for the stimulation of the nerves associated with carotid and aortic chemoreceptors and baroreceptors, aimed at inducing vasodilation and increasing cerebral perfusion in the brain of a living body.

The term “about” refers hereinafter to a range of 25% below or above the referred value.

The term “plurality” refers hereinafter to any integer greater than or equal to 2.

The term “fluid” refers hereinafter to a liquid or a gas.

The present invention provides a system for treating a medical condition in a living body of a patient, comprising (a) at least one implant, adapted to be retrievably implanted in a patient; the implant comprising at least one electrostimulation module comprising a proximal end and a distal end, the distal end comprising at least one first distal end member; and at least one electrically conductive electrode mounted in the at least one first distal end member; and (b) at least one electrical stimulation unit, adapted for producing an electrical waveform and connected to at least one of said electrodes, wherein the implant is implanted adjacent to at least one of the group consisting of: the carotid sinus nerve, the aortic nerve, the common carotid artery, the external carotid artery, internal carotid artery, carotid artery bifurcation, carotid body, aortic body, aortic arch receptors and any combination thereof within the living body; further wherein the electrical stimulation unit is maintained outside the patient's body and is adapted to program, generate, control and deliver the electrical waveform, delivery to the electrodes being via at least one of a group consisting of a wired connection and a wireless connection.

In an embodiment, the system comprises two main elements:

1. An Electrical Stimulation Unit (ESU) comprising a designated, programmable pulse generator capable of providing electrical stimulation regimes, and a designated Graphic User Interface Unit to select and load the stimulation program and monitor the actual electrical parameters. The ESU is reusable; it will be cleaned before each use and can be used in a sterile covering.

2. The stimulation system: A system including leads and electrodes to be inserted through the self-guiding catheter to the carotid bifurcation. The system or components thereof may be single use, supplied sterile. Many embodiments of the stimulation system also comprise an anchoring unit to keep the electrodes in position during treatment.

In embodiments of the system, the stimulation system stimulates at least one of the following: nerves of carotid body, the carotid sinus nerve (also called Hering's nerve), the aortic nerve, which is a branch of the vagus nerve, chemoreceptors in arteries adjacent to the bifurcation of the carotid, baroreceptors in arteries adjacent to the bifurcation of the carotid.

In an embodiment of the system, the stimulation catheter is a minimally invasive disposable catheter. The catheter may be single use, supplied sterile. In an endovascular approach, the catheter may be introduced under fluoroscopy via a femoral approach; other approaches may be used. The electrodes are positioned adjacent to the chemoreceptors (in the area of the carotid bifurcation). The catheter is then connected externally to the ESU and therapy parameters are set by the physician. The patient returns, with the stimulation system, to the Neuro-ICU for continued monitoring to determine when therapy can be discontinued and the catheter removed. At the end of the treatment the stimulation catheter is removed in the angiography lab.

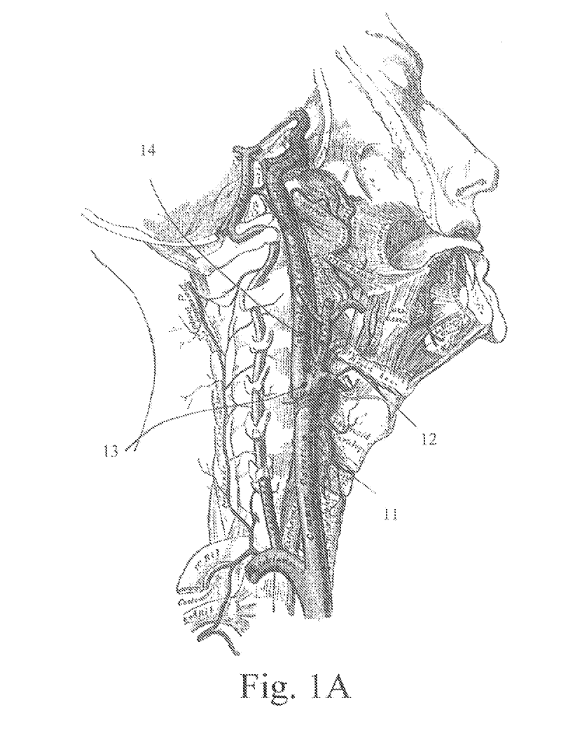

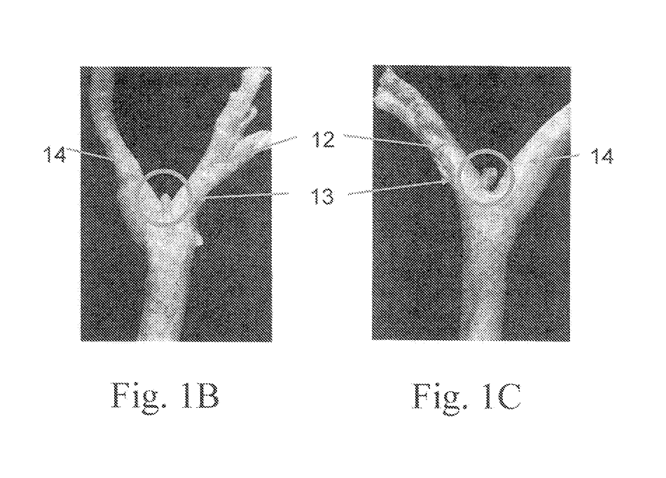

FIG. 1A is a general anatomic description, schematically depicting the major vascular structures of the right throat, neck and head, up to the temple region. Specifically, the figure depicts the common carotid artery that bifurcates into the internal carotid artery (14) and the external carotid artery (12), at a carotid bifurcation (13). FIG. 1B and FIG. 1C depict, respectively, the left (FIG. 1B) and right (FIG. 1C) carotid bodies, in positions and of sizes typical of about 95% of the population. The internal carotid artery (14), external carotid artery (12) and carotid bodies (circled, 13) are shown.

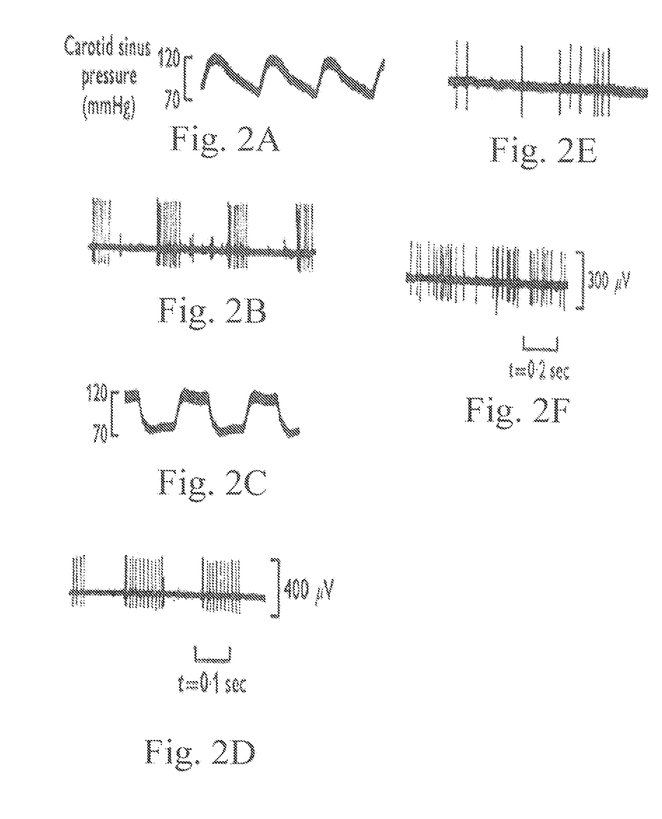

FIG. 2 depicts electrical discharge patterns from baroreceptor and chemoreceptor fibers; FIG. 2B depicts the discharge from a single baroreceptor fiber when the left carotid sinus is naturally perfused, as depicted in the pressure figure of FIG. 2A. FIG. 2D depicts the discharge from a single baroreceptor fiber when the left carotid sinus is artificially perfused, as depicted in the pressure figure of FIG. 2C. FIG. 2E depicts the discharge from a single chemoreceptor fiber when the left carotid sinus is perfused with arterial blood. FIG. 2F depicts the discharge from a single chemoreceptor fiber when the left carotid sinus is perfused with venous blood. Mean sinus pressure is 130 mmHg in both cases, and the respective average frequencies of discharge are 5 Hz and 18.5 Hz, respectively. The respective average frequencies of discharge were 33 impulse/s (2A and 2B) and 28 impulses (2C and 2D).

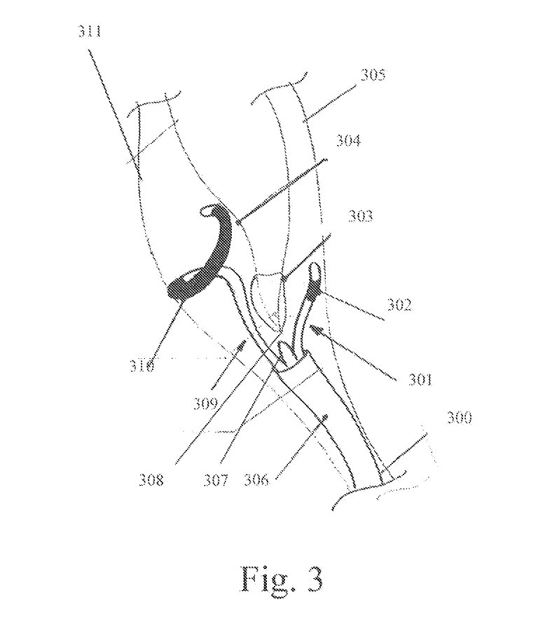

FIG. 3 schematically depicts a selected embodiment of the present invention. A multiple channel distal end (306) is endovascularly positioned near a carotid body (303) with the branching point of the module (307) being adjacent to the bifurcation of the carotid (308).

A first distal end member (301) is shown disposed within the external carotid artery (305). A second distal end member (309) is shown disposed within the internal carotid artery (311). One electrically conductive electrode (302) is shown on the first distal end member (301). One electrically conductive electrode (310) is shown on the second distal end member (309). The first and second distal end members (301 and 309, respectively) are used to stimulate a carotid baroreceptor and a carotid chemoreceptor. The sinus is shown (304) and the common carotid artery (300). In this embodiment, the shape of one distal end member (309) is spiral so as to optimize treatment by optimizing the shape of the electrode.

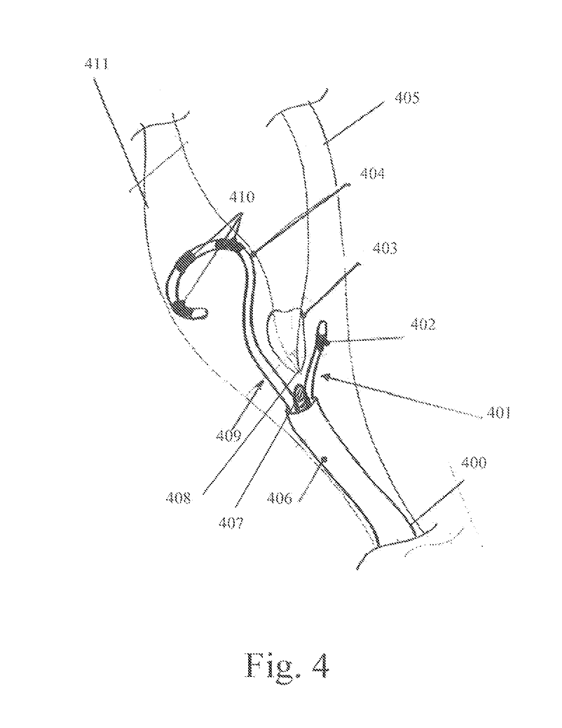

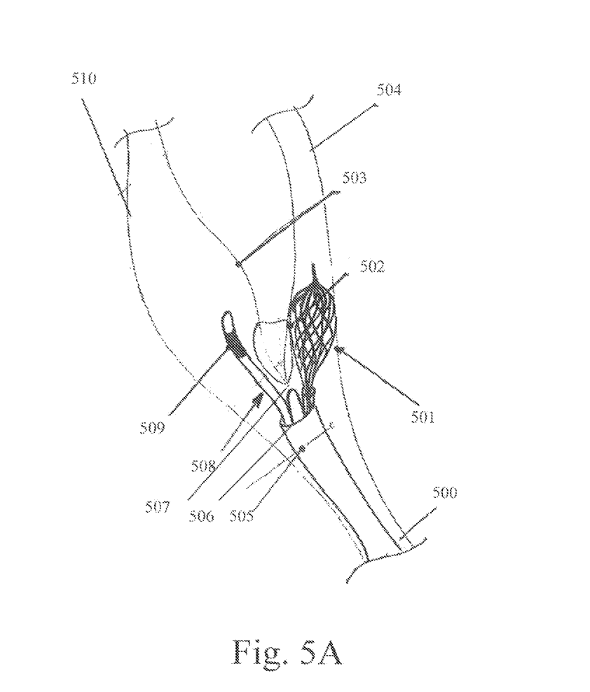

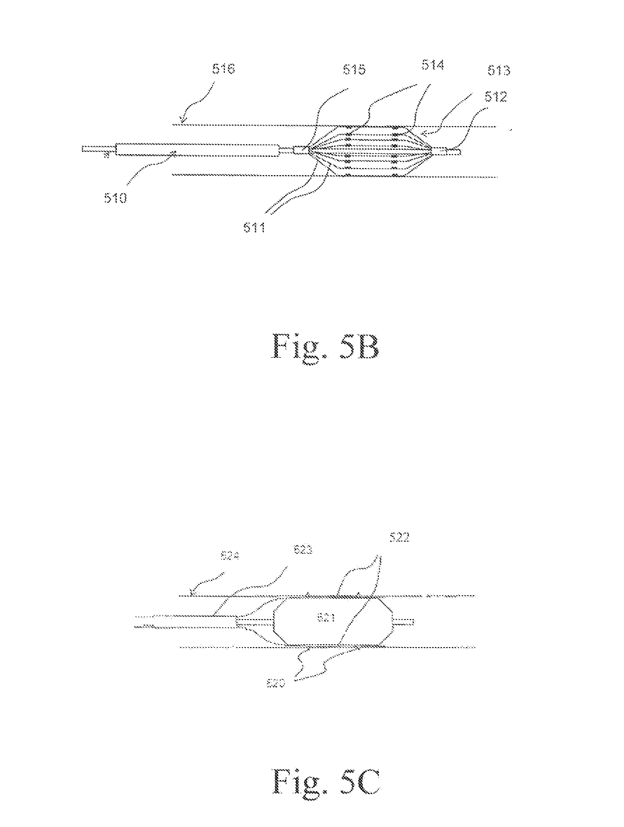

FIG. 4 schematically depicts a selected embodiment of the present invention. A multiple channel distal end (406) is endovascularly positioned near a carotid body (403) with the branching point of the module (407) being adjacent to the bifurcation of the carotid (408).

A first distal end member (401) is shown disposed within the external carotid artery (405).

A second distal end member (409) is shown disposed within the internal carotid artery (411). One electrically conductive electrode (402) is shown on the first distal end member (401). Three electrically conductive electrodes (410) are shown on the second distal end member (409). The first and second distal end members (401 and 409, respectively) are used to stimulate a carotid baroreceptor and a carotid chemoreceptor.

The sinus is shown (404) and the common carotid artery (400). In this embodiment, the shape of one distal end member (409) is spiral so as to optimize treatment by optimizing the positions of the electrodes.

It should be emphasized that the location of the electrodes (410) on the distal end member (409) and one relatively to the other, are optimize so as to minimize the distribution of the electric fields to anatomical locations other than at least one selected from a group consisting of: carotid baroreceptor(s), chemoreceptor(s), aortic arch receptors and any combination thereof.

Furthermore, the location of the electrodes (410) on the distal end member (409) and one relatively to the other are optimized so as to enhance the response of at least one selected from a group consisting of: carotid baroreceptor(s), chemoreceptor(s), aortic arch receptors and any combination thereof.

FIG. 5A to FIG. 5C schematically depict embodiments of the anchoring member of the present invention.

According to the present invention, the anchoring member ensures the best positioning of the end members with reference to the internal wall of the artery, so as to best stimulate the carotid baroreceptor(s), chemoreceptor(s), aortic arch receptors and any combination thereof.

FIG. 5A schematically depicts one embodiment of the entire mechanism with one embodiment of an anchoring member on one distal end member, whereas FIG. 5B and FIG. 5C schematically depict only the distal end member comprising the anchoring member and not the other parts of the present invention.

Reference is now made to FIG. 5A in which a multiple channel distal end (505) is endovascularly positioned near a carotid body (502).

A first distal end member (501) is shown disposed within the external carotid artery (504) with the branching point of the module (506) being adjacent to the bifurcation of the carotid (507).

A second distal end member (508) is shown disposed within the internal carotid artery (510).

One electrically conductive electrode (509) is shown on the second distal end member (508). In this particular embodiment, the first distal end member (501) serves as a electrically conductive electrode and as an endovascular anchoring member that is in the form of a cylindrical mesh positioned on the first distal end member. The first and second distal end members (501 and 508, respectively) are used to stimulate a carotid baroreceptor and a carotid chemoreceptor. The sinus is shown (503) and the common carotid artery (500).

It should be emphasized that according to one embodiment of the present invention, the endovascular anchoring member is provided in addition to the first (501) and second (508) distal end member.

FIG. 5B schematically depicts a selected embodiment of a single distal end member of the present invention.

The distal end (510) is endovascularly positioned in the carotid artery (516). In this particular embodiment, the anchoring mechanism (513) comprises a plurality of wires (511) in the form of a cage, a set of wires arranged radially and meeting at two common points, one at the proximal end (515) of the cage and one at the distal end (512) of the cage.

In this embodiment, each the wire serves as a conductive lead for two electrodes (514) so as to optimize treatment by optimizing the positions of the electrodes.

According to one embodiment of the present invention, the cage is characterized by at least two configurations; a collapsed state adapted to allow free longitudinal motion of the endovascular electrode inside a blood vessel lumen, and an expanded state, in which anchoring member is in contact with at least a longitudinal and an angular portion of the lumen.

Such contact provides better stimulation of the chemoreceptors and baroreceptors.

It should be emphasized that the endovascular cage is reversibly transitionable between the collapsed state and the radially expanded state.

FIG. 5C schematically depicts a selected embodiment of a single distal end member of the present invention.

The distal end (523) is positioned in the carotid artery (524). In this particular embodiment, the anchoring mechanism comprises a double-walled balloon (521) with a central lumen to permit blood flow. In this embodiment, the balloon supports conductive leads (522) attached to electrodes (520) so as to optimize treatment by optimizing the positions of the electrodes.

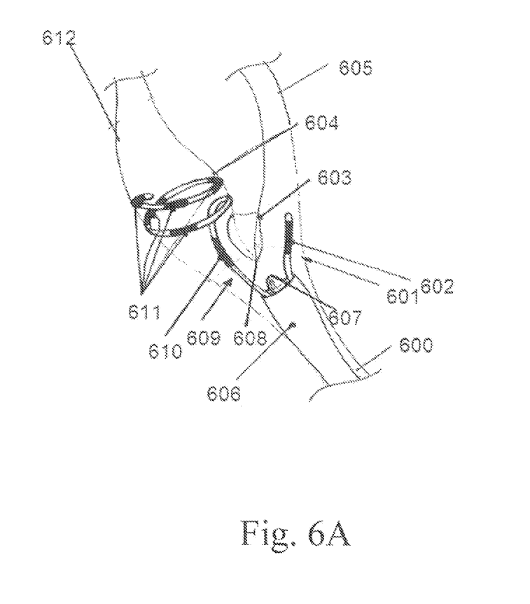

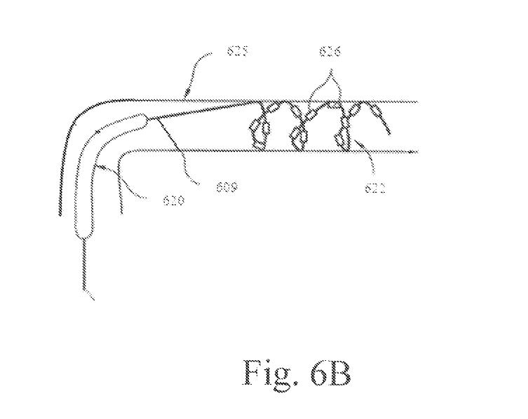

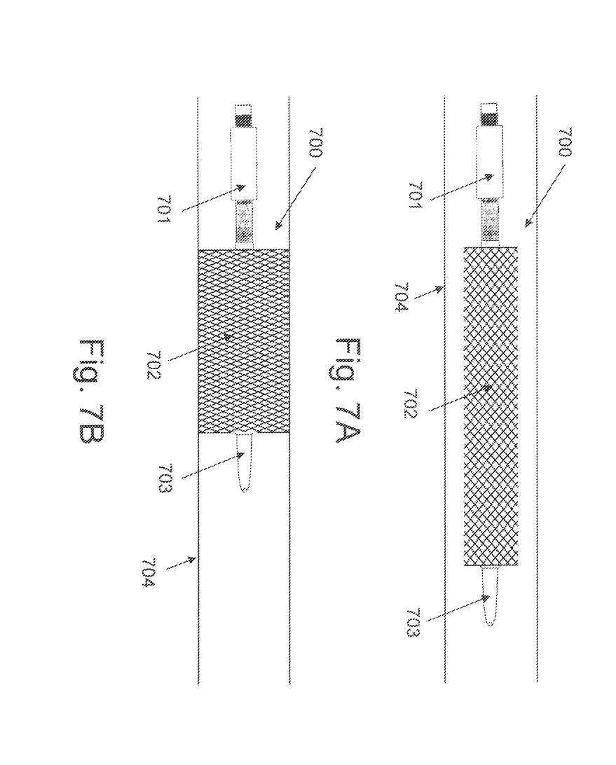

FIG. 6A to FIG. 6B schematically depict embodiments of optimization mechanisms to ensure a best stimulation of the chemoreceptors, baroreceptors, carotid aortic arch receptors and any combination thereof.

FIG. 6A schematically depicts one embodiment of the entire mechanism with one embodiment of an anchoring member on one distal end member, whereas FIG. 6B schematically depicts only the distal end member comprising the anchoring member and not the other parts of the present invention.

Schematically depicted in FIG. 6A is a multiple channel distal end (606) endovascularly positioned near a carotid body (603) with the branching point of the module (607) being adjacent to the bifurcation of the carotid (608).

A first distal end member (601) is shown disposed within the external carotid artery (605).

A second distal end member (609) is shown disposed within the internal carotid artery (608). One electrically conductive electrode (602) is shown on the first distal end member (601).

A plurality of electrically conductive electrodes (610, 611) are shown on the second distal end member (609).

In this particular embodiment of the present invention, the first and second distal end members (601 and 609, respectively) are used to stimulate a carotid baroreceptor and a carotid chemoreceptor.

The sinus is shown (604) and the common carotid artery (600). In this embodiment, the shape of the distal end member (609) is spiral so as to optimize treatment by optimizing the shape and position of the electrodes.

It should be emphasized that the shape of the distal end member (609) is selected so as to enhance the response of at least one selected from a group consisting of: carotid baroreceptor(s), chemoreceptor(s), aortic arch receptors and any combination thereof.

Furthermore, the location of each of the electrodes (611) and the distance between each of the electrodes (611) are optimized.

FIG. 6B schematically depicts a selected embodiment of a single distal end member of the present invention.

The distal end (620) is endovascularly positioned in a carotid artery (625) and comprises a distal end member (609) plurality of electrically conductive electrodes (626) disposed along it.

In this embodiment, the shape of the distal end member (609) is spiral (622) and the distance between each pair of electrodes (626) are short relative to the radius of curvature of the spiral so as to optimize treatment by optimizing the position of the electrodes.

FIG. 7 schematically depicts a distal end member according to an embodiment of the anchoring mechanism.

FIG. 7A schematically depicts the anchoring mechanism (700) in an artery (704) in the collapsed state. The anchoring mechanism comprises a base (701) which is connected to a mesh (702), to which a tip (703) is connected.

In the collapsed state, the mesh (702) is adapted to allow free longitudinal motion of the endovascular electrode inside a blood vessel lumen (704).

FIG. 7B schematically depicts the anchoring member (700) in an artery (704) in the expanded state.

In the expanded state, the tip (703) of the distal end member (701) has been pulled back towards the base (701) of the distal end member so that the mesh (702) has been expanded radially relative to its position in the collapsed state. In the expanded state the same is adapted to engage at least a longitudinal and an angular portion of the lumen.

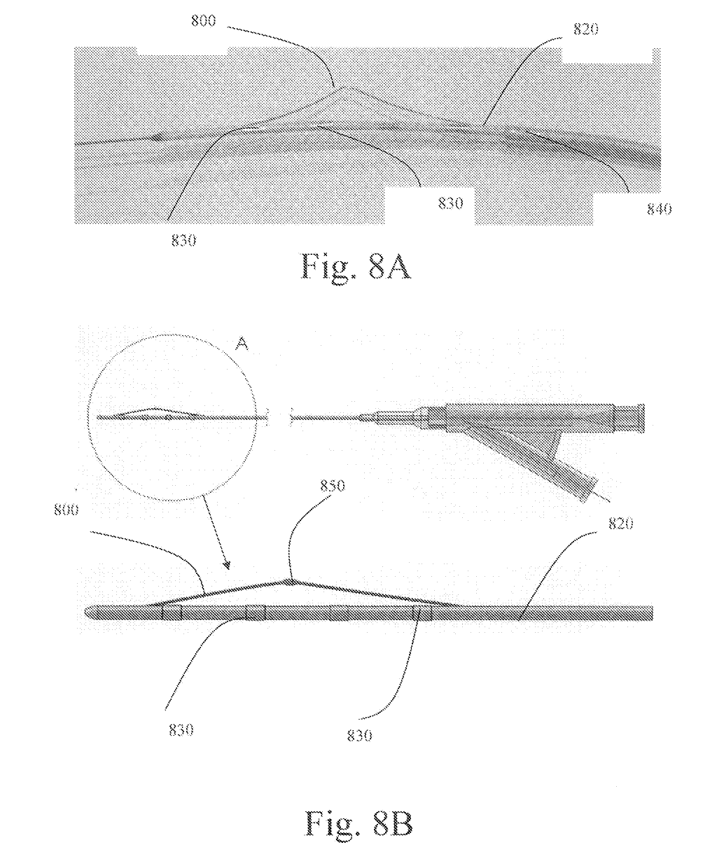

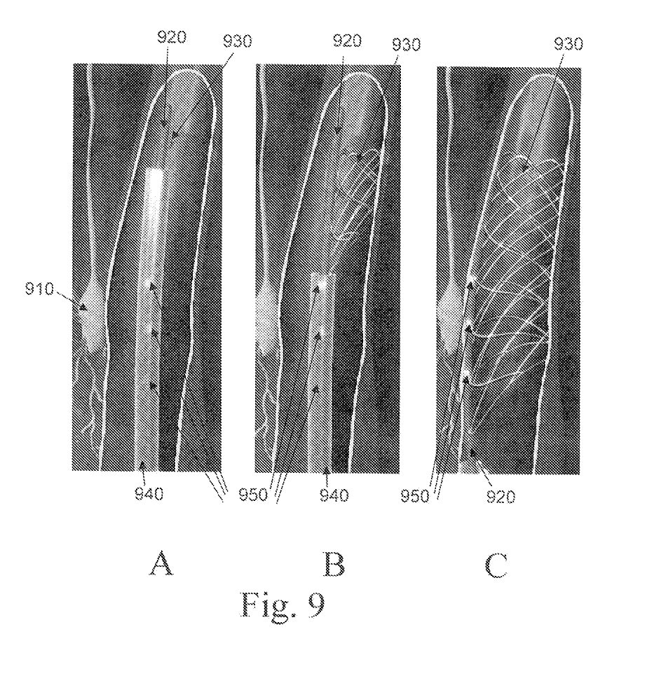

Reference is now made to FIG. 8A, illustrating another embodiment of the anchoring member (800).

According to this embodiment, the anchoring member (800) is coupled to the at least one of the distal end member 820, (upon which at least one electrode 830 is disposed).

According to this embodiment, the anchoring member (800) is characterized by having spring-like mechanical properties and has at least 2 configurations: (a) closed (collapsed) configuration, in which the anchoring member (800) is enclosed within channel 840 and have a substantially linear/planar configuration; and, (b) a deployed (expanded) configuration, in which the anchoring member (800) is pushed out of channel 840 (or alternatively channel 840 is withdrawn backwardly). In the deployed configuration, the anchoring member (800) assumes a ‘bent’ configuration.

According to one embodiment, the anchoring member (800) is made of an elastic and flexible material so as to have spring-like mechanical properties.

Prior to the deployment of the distal end member 820, the same is maintained within channel 840. In this position, the anchoring member 800, is enclosed within channel 840 and maintains a substantially planar (and/or linear) configuration (limited by channel's 840 diameter).

Once the desired location is reached (e.g., reaching the chemoreceptors, baroreceptors, aortic arch receptors and any combination thereof), channel 840 is withdrawn backwards; thus, (i) exposing the distal end member; (ii) the anchoring member 800 is exposed from channel 840 and is bent.

Reference is made again to FIG. 8A which illustrates the position of the anchoring member 800 once the same is exposed out from channel 840 and resumes its bent configuration.

In the deployed configuration, the anchoring member 800, at least partially engages with at least a longitudinal and an angular portion of the blood lumen/vessel, so as to fixate and anchor the distal end member and especially the electrodes to a desired position/location.

The deployment of the anchoring member 800 to the expanded configuration ensures the desired positioning of the distal end member and eventually the electrodes.

A desired positioning of the electrodes could be e.g., the positioning of the electrodes adjacently and in proximity to the blood vessel's internal wall.

Reference is now made to FIG. 8B illustrating another embodiment of the anchoring member 800. According to this embodiment, the anchoring member 800 additionally comprises at least one RF-opaque portion 850.

It should be pointed out that a material is called RF-opaque if it blocks, reflects, and scatters RF waves.

Such an RF-opaque portion 850 is used to allow visualization by electromagnetic imaging modalities (such as fluoroscopy). In this manner, the operator can monitor the location of the electrode.

Reference is now made to FIG. 9, showing another embodiment of the anchoring member. In this embodiment, the anchoring member is of stent-like form, and is of sufficiently stiff or spring like material and of appropriate design such that it will transition to an expanded state unless restrained therefrom. A schematic illustration of such a design is shown (930) in FIG. 9C.

In this embodiment, an endovascular approach is used. The delivery catheter (940) is positioned near the carotid body (910) and an overtube is withdrawn backwards to expose the electrodes (950) and stent-like anchoring member (930). In FIG. 9A, the device is shown as positioned in the desired location prior to the start of the withdrawal of the delivery catheter. The stent-like anchoring member (930) is shown in a position where it is fully in the collapsed position within the overtube. In FIG. 9B, the overtube has been partly withdrawn and the stent-like anchoring member is partly expanded. It is touching the wall of the artery (shown in cutaway view) but has not yet begun to press the electrodes against the artery wall. In FIG. 9C, the overtube has been fully withdrawn and the stent-like anchoring member is fully expanded. The electrodes are pressed against the artery wall in the region of the carotid body (950). Removal of the device comprises a reversal of the process of insertion. The overtube is pushed forward, transitioning the stent-like anchoring member from the expanded state (FIG. 9C) through the partly-expanded state (FIG. 9B) to the collapsed state (FIG. 9A). The device is now entirely within the overtube, is free to move longitudinally within the artery and can be repositioned or removed from the body.

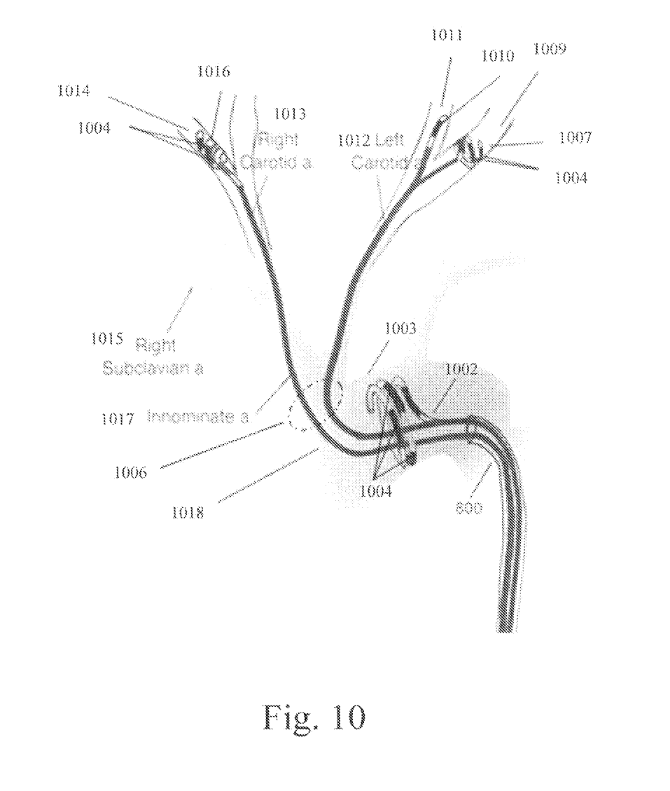

FIG. 10 schematically depicts a system for stimulation of chemoreceptors and baroreceptors in both carotid bifurcations and in the aortic arch. In this system, the base of the distal end (1000) is disposed within the aorta (1001).

The distal end (1000) subdivides adjacent to the arch of the aorta (1003). In this embodiment, one subdivision forms a spiral aortal distal end (1002) endovascularly placed in the arch of the aorta (1003). The spiral aortal distal end is an endovascular anchoring device with a plurality of electrically conductive electrodes (1004) disposed along it. In this embodiment, the shape of the distal end member (1002) is spiral so as to optimize treatment by optimizing the position of the electrodes.

The second subdivision (1018) enters the innominate artery (1017) and subdivides adjacent to the branching point (1006) where the left carotid artery divides from the innominate artery (1017).

The subdivision within the left carotid artery (1005) further subdivides adjacent to the branching point where the left internal carotid artery (1011) and the left external carotid artery (1009) divide.

The subdivision in the left external carotid (1009) forms a spiral distal end member (1007) endovascularly placed in the left external carotid (1009).

The spiral distal end is an endovascular anchoring device with a plurality of electrically conductive electrodes (1004) disposed along it. In this embodiment, the shape of the end member (1007) is spiral so as to optimize treatment by optimizing the position of the electrodes (1004).

The subdivision (1012) in the left internal carotid (1011) has a single electrode (1010) disposed on it.

The subdivision (1018) in the innominate artery (1017) passes into the right carotid artery (1013) and from the right carotid artery (1013) into the right internal carotid artery (1016).

There it forms a spiral distal end member (1014) endovascularly placed in the right internal carotid (1016).

The spiral distal end is an endovascular anchoring device with a plurality of electrically conductive electrodes (1004) disposed along it. In this embodiment, the shape of the distal end member (1014) is spiral so as to optimize treatment by optimizing the position of the electrodes. In this embodiment, no distal end member has been placed in the right external carotid artery (1013).

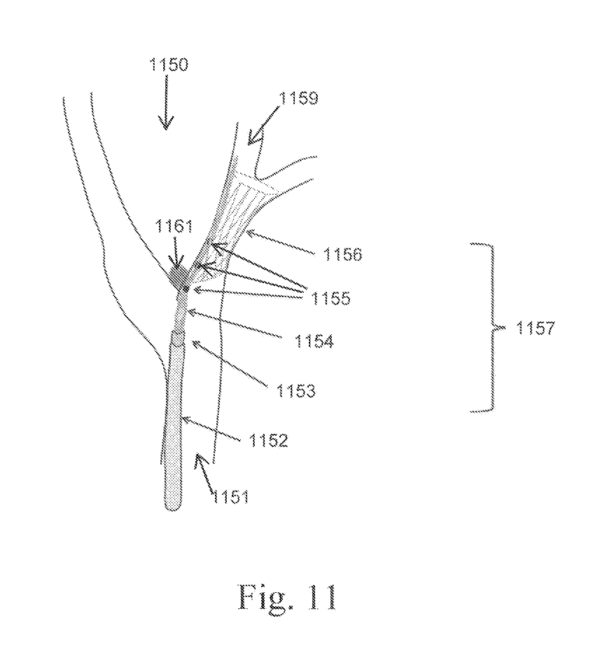

Reference is now made to FIG. 11 schematically illustrating an embodiment a system (1150) for stimulation of chemoreceptors and baroreceptors in one carotid artery. In this embodiment, the device is inserted intravascularly via the femoral artery. A delivery catheter (1152) is inserted via the femoral artery into the common carotid artery (1151). The delivery catheter (1152) contains an over tube (1153) surrounding the stimulation module (1157) and the fixation element (1156) is then inserted through the delivery catheter into the carotid bifurcation and the external carotid artery. The stimulation module (1157) is positioned and the overtube is retrieved to expose the electrodes and fixation element. The stimulation module (1157) comprises a catheter (1154), wires (not shown) and electrodes (1155), and it is held in position in the external carotid artery (1159) near the base of the carotid body (1161) by an anchoring member (1156).

The electrodes can be inserted either endovascularly or extravascularly. In an endovascular approach, a catheter is inserted percutaneously into an artery. Once the catheter is in position, the electrodes are exposed, in a position adjacent to the carotid body, as described hereinabove, and the anchoring member is transitioned into a configuration which retains the electrodes in position.

In an extravascular approach, the electrodes and anchoring member are positioned proximate to the carotid body within the carotid bifurcation region, but are not positioned within an artery. In the extravascular approach, there are two main methods of emplacing the electrodes and anchoring member: a. Percutaneous entry via the neck, and b. Surgically opening the neck and exposure of the carotid bifurcation. In an extravascular approach, there is no entry into the artery.

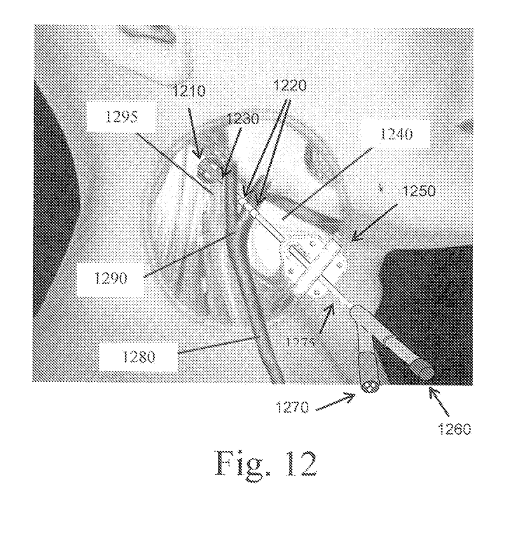

In reference to FIG. 12, this drawing (1200) serves as an example of an extravascular approach. In an extravascular approach, each electrostimulation module (1275) is inserted and positioned so that the electrodes (1220) are near the carotid bifurcation (1290). In this example, the approach can be via percutaneous insertion or surgical exposure. A needle (not shown) is used to insert and position the implant. A balloon catheter (1230) with distal balloon (1210) is an example of an internal fixation element. The balloon catheter (1230) is inserted within the electrostimulation module and is connected to an external inflation port (1260). The balloon (1210) is placed distal to the carotid bifurcation (1290) and is inflated with fluid. The clamp (1250) is an example of external fixation. The electrostimulation module (1275) is connected to an electrical connector (1270) and, via the connector (1270), to the electrical stimulation unit (ESU) (not shown). The ESU generates an electrical signal that is transmitted to the electrodes (1220) via wiring, which generates an electrical field in the area of the carotid body, as discussed hereinabove. For removal of the device, the balloon (1210) is deflated by removal of the inflation fluid. The jugular vein (1295) and the hyoid bone (1240) are shown for reference.

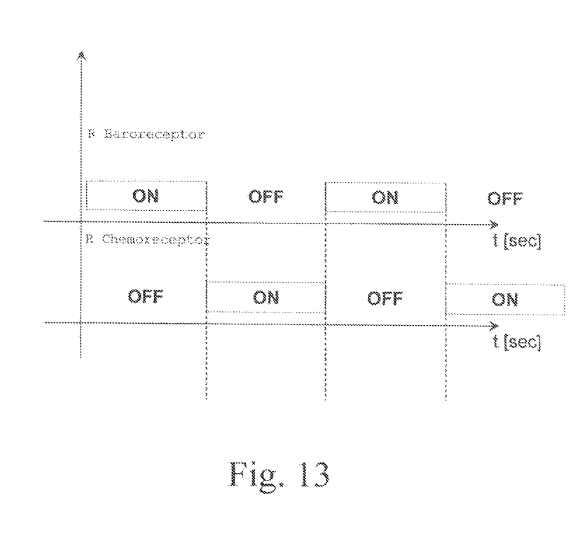

FIG. 13 schematically depicts an intermittent unilateral stimulation regimen for the right baroreceptor and right chemoreceptor. In this selected embodiment of the present invention, either the right baroreceptor or the right chemoreceptor is activated. The two abovementioned receptors are not activated simultaneously.

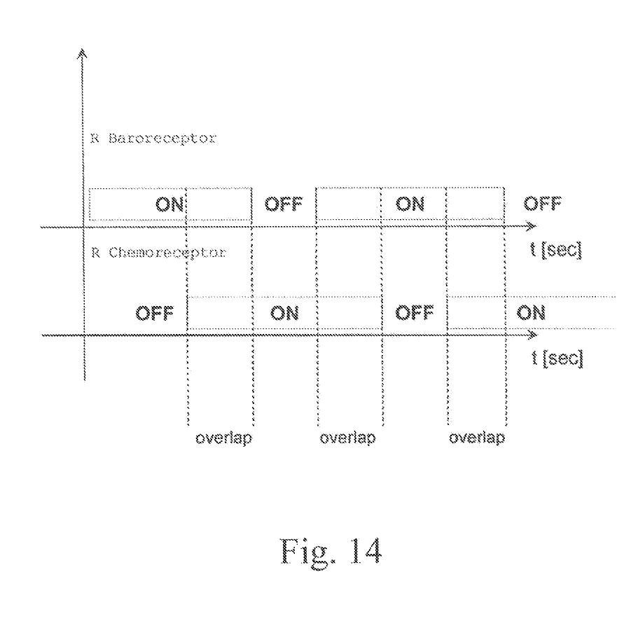

FIG. 14 schematically depicts an intermittent unilateral stimulation regimen for the right baroreceptor and right chemoreceptor. In this selected embodiment of the present invention, the right baroreceptor or the right chemoreceptor is activated in a partially overlapping mode. In this embodiment, there are times in which each of the receptors is activated alone, and times in which the two receptors are activated in tandem.

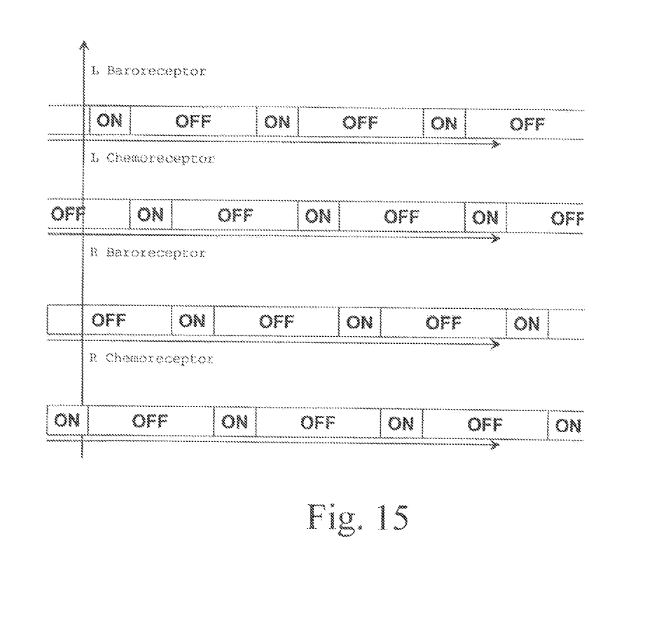

FIG. 15 schematically depicts an intermittent bilateral stimulation regimen for the right and left baroreceptors and for the right and left chemoreceptors. In this selected embodiment of the present invention, each of the four abovementioned receptors is activated alone, with the remaining three receptors left non-activated.

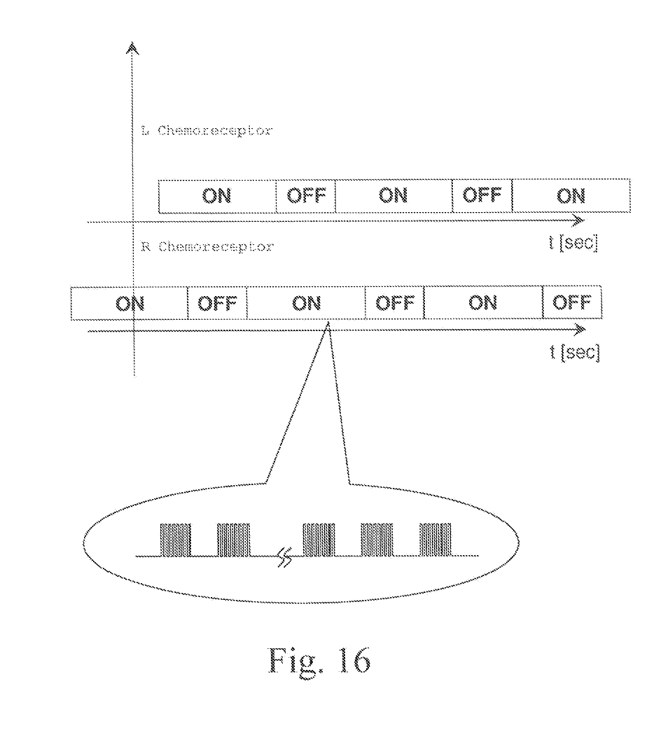

FIG. 16 schematically depicts an intermittent bilateral stimulation regimen for the carotid artery right chemoreceptor and the left chemoreceptor. In this selected embodiment of the present invention, the right chemoreceptor or the left chemoreceptor is activated in a partially overlapping mode. Namely, there are times in which each of the chemoreceptors is activated alone, and times during which both of the chemoreceptors is activated. The balloon schematically depicts an example of an active stimulation period, which is comprised of a uniphasic pulse train, spaced by electrically-inactive periods that are intended to overcome neurological and biological tolerance to the stimulation regimen.

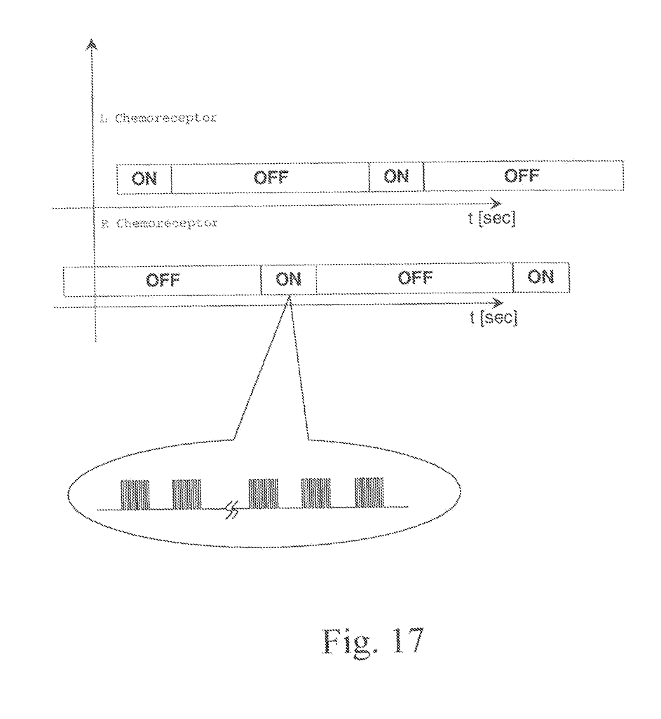

FIG. 17 schematically depicts an intermittent bilateral stimulation regimen for the carotid artery right chemoreceptor and the left chemoreceptor. In this selected embodiment of the present invention, the right chemoreceptor or the left chemoreceptor is activated in a non overlapping mode. Namely, there are times in which each of the chemoreceptors is activated alone, and times during which neither of the two chemoreceptors is activated. The balloon schematically depicts an example of an active stimulation period, which is comprised of a uniphasic pulse train, spaced by electrically-inactive periods that are intended to overcome neurological and biological tolerance to the stimulation regimen.

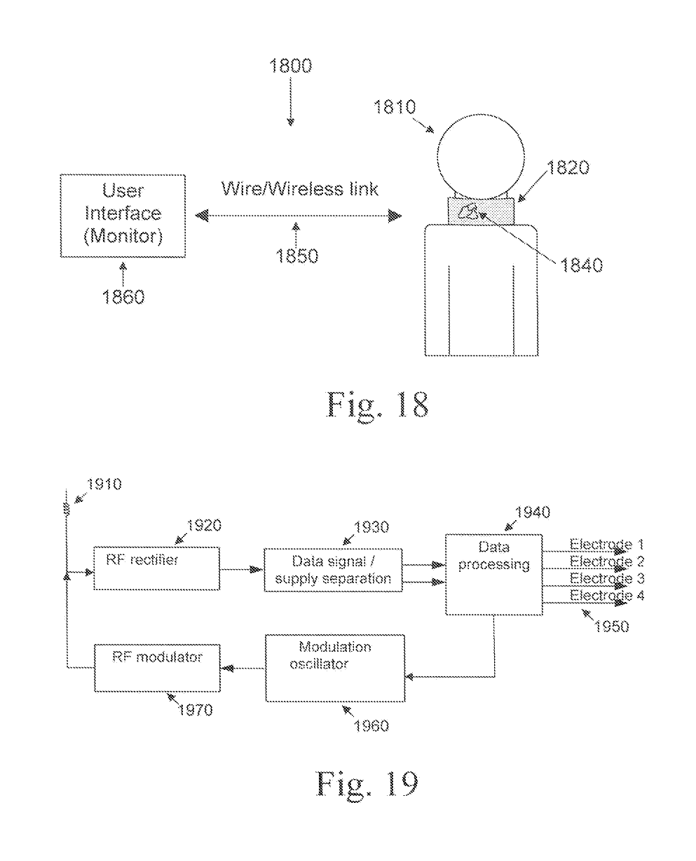

In reference to FIG. 18, in some embodiments of the system (1800), the implant, which is in the region of the carotid bifurcation, is connected to an External Unit which is subsystem of the Electrical Stimulation Unit (ESU) and which is built into an ergonomic device (1820), such as a belt or collar, worn on the body of the patient (1810). The External Unit receives commands from the user interface, which is also a subsystem of the ESU (1860), and transmits electrical energy to the implant or implants (1840) in response to the commands. The External Unit receives feedback from the implant (1840) and inputs from other sensors, and transmits the information to the user interface (1860). The External Unit is capable of individually controlling each implant (1840), if there is more than one, and of individually controlling each of the electrodes in any given implant so that full control of the duration, magnitude and location of the electrical stimulation is enabled. The control may be from a location remote from the patient, for non-limiting example from an adjacent room, especially if a wireless link is used. The connection between the implant (1840) and the External Unit can be wired or wireless (1830). The External Unit is connected via a link (1850), either wired or wireless, with a user interface/monitor/controller (1860). The ergonomic device (1820) can be worn in any convenient position on the body, for example, around the neck as a collar, around the arm, on a finger, around the chest or waist, around the thigh or leg, or even around the head as a headband. FIG. 18 shows the External Unit (1820) connected wirelessly to the implant (1840), and wirelessly (1850) to the user interface/controller (1860), which has a monitor to enable the patient or the caregiver to monitor the treatment and modify it when necessary.

An example of the functioning of the External Unit is shown in FIG. 19. In this embodiment, a wireless link is used. The External Unit receives and transmits signals to the user interface (not shown) via an antenna (1910). The received signal passes through a rectifier (1920) which removes the carrier wave, from the received signal. The DC signal is then separated (1930) into a data signal and a supply of electrical energy. The data signal and power supply are then passed to a data processor (1940) which determines the pulse scheduling, voltages, and currents to be applied by the electrodes (4 in this example), and applies the currents and voltages as scheduled to the electrodes (1950) implanted near the targeted anatomical location (not shown). The data processor receives feedback from the electrodes (1950) and from any sensors in the network (not shown). The data processor may also provide other information on the operation of the implants. All these signals are passed to a modulation oscillator (1960) and an RF modulator (1970), which transform the signals to an appropriate format for wireless transmission. The transformed signals are transmitted to the user interface via the antenna 1910.

### EXAMPLES

Examples are given in order to demonstrate the embodiments claimed in the present invention. These examples, which are a pre-clinical test, describe the manner and process of the present invention and set forth the best mode contemplated by the inventors for carrying out the invention, but are not to be construed as limiting the invention.

### Example 1

### Vasodilatory Effect Following Electrostimulation

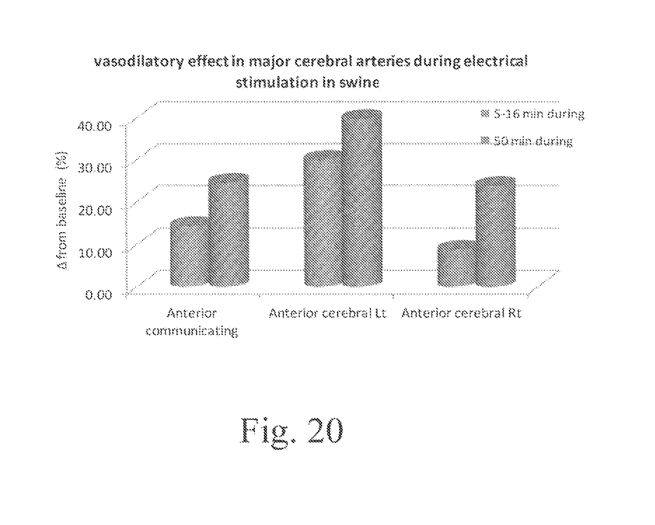

Reference is now made to FIG. 20, which schematically illustrates the vasodilatory effect in the major cerebral arteries of electrical stimulation of the carotid body in swine. Vessel diameter increases significantly in the anterior communicating artery, and the anterior cerebral left and right arteries. In this example the anterior cerebral right artery diameter increases only by more than 5% during the period for 5 to 16 minutes after the start of treatment and rises to more than 20% during the subsequent 50 minutes of treatment. The anterior communicating artery has a similar response to this stimulation, with vessel diameter increasing by more than 10% during the period for 5 to 16 minutes after the start of treatment and by more than 20% during the subsequent 50 minutes of treatment. The anterior cerebral left artery shows significantly better response, with vessel diameter increasing by nearly 30% during the period for 5 to 16 minutes after the start of treatment and by nearly than 40% during the subsequent 50 minutes of treatment.

### Example 2

### Electro-Stimulation of the Chemoreceptors

The following example illustrates the in-vivo implantation of lead electrodes in the internal carotid arteries (left and right), in swine and the effect of the stimulation on arterial blood pressure (BP) and cerebral perfusion (CP), measured using Laser Doppler).

A delivery with a multiple electrode catheter was emplaced in the internal carotid arteries, near the carotid bifurcation, the electrical stimulation signal was delivered and the physiological effect was measured.

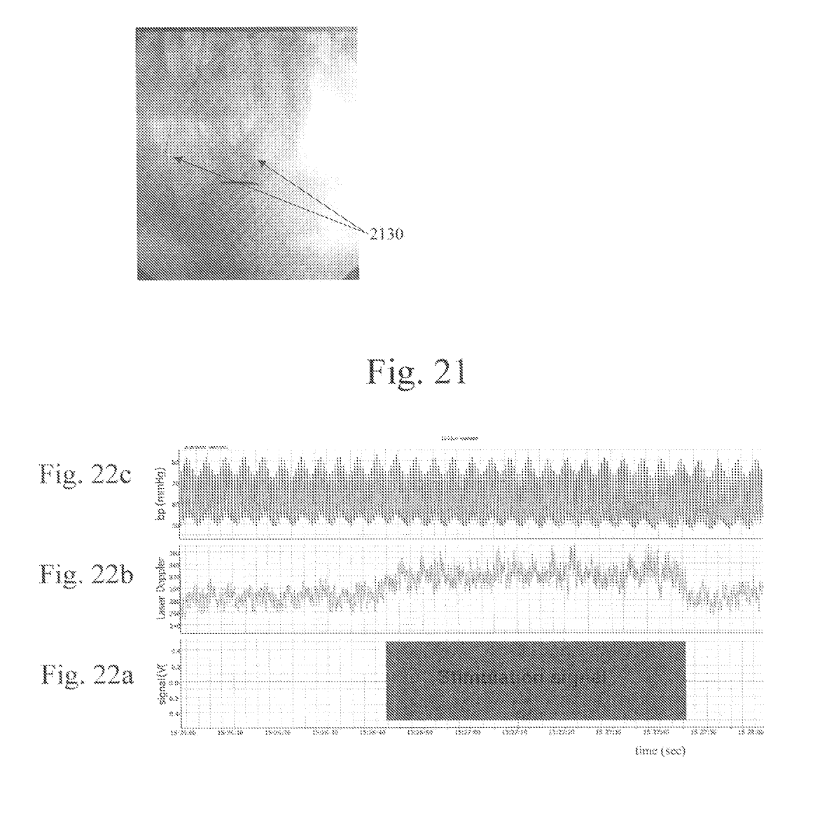

Reference is now made to FIG. 21 illustrating the positioning of the electrodes 2130 in the internal carotid arteries in swine.

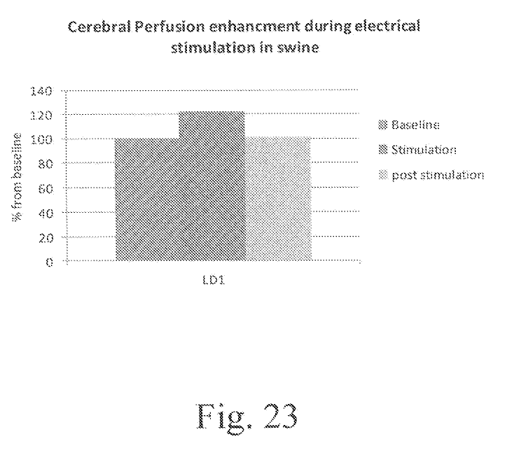

Reference is now made to FIGS. 22a-22c illustrating the test results.

Reference is now made to FIG. 22a which illustrates the electrical signal being applied vs. time.

The influence of the applied signal is seen in FIG. 22b and FIG. 22c.

It should be pointed out that FIG. 22a-22c are all provided on a unified time scale so as to see the instant effect of the signal application.

As can be seen in FIG. 22b the bilateral carotid body stimulation (at the carotid bifurcation) led to an instant and continuous 12% increase in cerebral perfusion (as measured by Laser Doppler).

### Example 3

### Cerebral Perfusion Enhancement Following Electrostimulation

Reference is now made to FIG. 23, which shows the enhancement of cerebral perfusion in swine during electrical stimulation. Enhancement of cerebral perfusion will be different from vasodilation, because perfusion enhancement depends on factors other than vasodilation, such as blood pressure. During stimulation, cerebral perfusion increases by more than 20% over the baseline value, returning to the baseline value after the end of the stimulation.

In summary, the present invention provides an electrostimulation system that enables dilatation of cerebral blood vessels and enhancement of cerebral perfusion when the carotid bodies in the area of the carotid artery bifurcation are stimulated.

1. A system for treating a medical condition in a living body of a patient, comprising:
a. at least one implant, adapted to be retrievably implanted in said patient; said implant comprising: at least one electrostimulation module comprising a proximal end and a distal end, said distal end comprising at least one first distal end member; and at least one electrically conductive electrode mounted in said at least one first distal end member; and
b. at least one electrical stimulation unit, adapted for producing an electrical waveform and connected to at least one of said electrodes
wherein said implant is implanted adjacent to at least one of the group consisting of: the carotid sinus nerve, the aortic nerve, the common carotid artery, the external carotid artery, internal carotid artery, carotid artery bifurcation, carotid body, aortic body, aortic arch receptors and any combination thereof within said living body; further wherein said electrical stimulation unit is maintained outside said patient's body and is adapted to program, generate, control and deliver said electrical waveform, said delivery to said electrodes being via at least one of a group consisting of a wired connection and a wireless connection.

2. The system according to claim 1, wherein the electrode of the implant is positioned such that an electrical excitatory waveform generated by said electrical stimulation unit is adapted to at least concurrently increase at least one of a group consisting of: cerebral perfusion and cerebral blood flow in a region in said subject's brain by more than about 7%, while changing mean arterial blood pressure by less than about 10%.

3. The system according to claim 1, wherein the electrode of the implant is positioned such that an electrical excitatory waveform generated by said electrical stimulation unit is adapted to at least concurrently increase at least one of a group consisting of: cerebral perfusion and cerebral blood flow in a region in said subject's brain by more than about 12%, while changing mean arterial blood pressure by less than about 7%.

4. The system according to claim 1, wherein the electrode of the implant is positioned such that an electrical excitatory waveform generated by said electrical stimulation unit is adapted to at least concurrently increase at least one of a group consisting of: cerebral perfusion and cerebral blood flow in a region in said subject's brain by more than about two times the percentage increase in mean arterial blood pressure.

5. The system according to claim 1, wherein the electrode of the implant is positioned such that an electrical excitatory waveform generated by said electrical stimulation unit is adapted to at least concurrently increase at least one of a group consisting of: cerebral perfusion and cerebral blood flow in a region in said subject's brain by more than about four times the percentage increase in mean arterial blood pressure.

6. The system according to claim 1, wherein said implant is configured to be implanted by at least one means selected from a group consisting of: endovascular means, extravascular percutaneous means and extravascular surgical means.

7. The system according to claim 6, wherein said endovascular means comprises a delivery catheter configured to deliver, position and retrieve the implant.

8. The system according to claim 7, wherein said delivery catheter is inserted through an insertion sheath.

9. The system according to claim 7, wherein said delivery catheter is inserted through a guiding catheter.

10. The system according to claim 1, wherein said electrical stimulation unit is configured to be externally disposed on said body of said patient.

11. The system according to claim 10, wherein said electrical stimulation unit is configured and shaped as at least one selected from the group consisting of: a belt, necklace, collar, bracelet, armlet, anklet, ring and any combination thereof.

12. The system according to claim 10, wherein said electrical stimulation unit is located around the neck of said patient.

13. The system according to claim 1, wherein said electrical stimulation unit comprises at least one antenna.

14. The system according to claim 1, wherein said implant additionally comprises at least one transmitter and receiver.

15. The system according to claim 14, wherein said transmitter is adapted to transmit feedback signals to said electrical stimulation unit.

16-98. (canceled)
